(19) United States
(12) Patent Application Publication
(10) Pub. No.: US 2022/0326229 A1
(43) Pub. Date: Oct. 13, 2022

(54) ASSAY FOR REAL-TIME SIMULTANEOUS RECRUITMENT OF ARRESTIN ISOFORMS TO G PROTEIN-COUPLED RECEPTORS

(71) Applicant: Purdue Research Foundation, West Lafayette, IN (US)

(72) Inventors: Richard M. van Rijn, Fremont, CA (US); Alexander R. French, West Lafayette, IN (US); Yazan Jamal Ibrahim Meqbil, West Lafayette, IN (US)

(73) Assignee: Purdue Research Foundation, West Lafayette, IN (US)

(21) Appl. No.: 17/591,461

(22) Filed: Feb. 2, 2022

Related U.S. Application Data
(60) Provisional application No. 63/170,057, filed on Apr. 2, 2021.

(51) Int. Cl. G01N 33/542; G01N 33/566

(52) U.S. Cl. CPC G01N 33/542; G01N 33/566; G01N 2333/726; G01N 2500/10

(57) ABSTRACT

Assay for real-time simultaneous recruitment of arrestin isoforms (e.g., β-arrestin), to a receptor, such as a G protein-coupled receptor (e.g., DOR); a biosensor; a bioarray; and a kit.

### CROSS-REFERENCE TO RELATED APPLICATIONS

This application claims priority to U.S. provisional patent application 63/170,057,filed Apr. 2, 2021, which is hereby incorporated by reference in its entirety.

### STATEMENT OF GOVERNMENT SUPPORT

This invention was made with government support under R01AA025368 and 5F32MH115432 awarded by the National Institutes of Health. The government has certain rights in the invention.

### REFERENCE TO A SEQUENCE LISTING SUBMITTED VIA EFS-WEB

The content of the ASCII text file of the sequence listing (named “69275-02SeqListing_13APR2022_ST25.txt” which is 76 kb in size, created on Apr. 13, 2022, and electronically submitted via EFS-Web on Apr. 14, 2022) is incorporated herein by reference in its entirety. The information recorded in computer readable form is identical to the written Sequence Listing provided herein (on paper) pursuant to 37 C.F.R. § 1.821(f).

### TECHNICAL FIELD

The present disclosure relates to the real-time assay of simultaneous arrestin isoform recruitment to a receptor.

G-protein coupled receptors (GPCRs) constitute the largest and most diverse group of eukaryotic membrane receptors. GPCRs are present in animals, plants, fungi and protozoa. Humans have almost 1,000 different GPCRs, and each one is highly specific fora particular signal, which can range from light to peptides, proteins, lipids, and sugars. Of 800 GPCRs that have been identified, about half have sensory functions, such as olfaction (˜400), taste (33), light perception (10) and pheromone signaling (5) (Mombaerts, Nat RevNeurosci 5(4): 263-278 (2004)). It is estimated that one-third to one-half of all available drugs bind to GPCRs. The remaining ˜350 non-sensory GPCRs mediate signaling by ligands that range in size from small molecules to peptides to proteins and are targets for the majority of drugs in clinical use (Overington et al., Nat Rev Drug Discov 5(12): 993-996 (2006); and Rask-Andersen et al., Annu Rev Pharmacol Toxicol 54: 9-26 (2014)), although only a minority of these receptors are exploited therapeutically.

Most human GPCRs can be grouped into five main families, as follows: glutamate, rhodopsin, adhesion, frizzled/taste2, and secretin. This system of classification is referred to as “GRAFS.” The rhodopsin family is the largest and comprises four groups, as follows: α, β, γ and δ. There are 13 sub-groups.

GPCRs consist of a single polypeptide that is folded into a globular shape and embedded in the plasma membrane of a cell with an extracellular N-terminus and an intracellular C-terminus. Seven hydrophobic transmembrane domains (TM1-TM7) span the entire width of the plasma membrane; this is why GPCRs are also referred to as seven-transmembrane receptors. TM1-TM7 are linked by three extracellular loops (ECL1-ECL3) and three intracellular loops (ICL1-ICL3). The extracellular loops form part of the pockets in which signal molecules bind.

The GPCR interacts with a G protein in the plasma membrane. The G protein is trimeric—having α, β and γ subunits. The α and γ subunits are attached to the plasma membrane by lipid anchors.

When there is no signal molecule present, the α subunit of the G protein binds guanosine diphosphate (GDP) and the resultant G-protein-GDP complex binds a nearby GPCR. This arrangement persists until a signal, such as a signaling molecule, binds the GPCR and causes it to undergo a conformational change. The conformational change in the GPCR activates the G protein, which replaces the GDP with guanosine triphosphate (GTP).

The G protein then dissociates into two parts: an α-subunit-GTP complex and a β-subunit—γ-subunit dimer. Both parts remain anchored to the plasma membrane but neither part is bound to the GPCR. Consequently, the parts diffuse laterally within the plasma membrane and interact with other membrane proteins.

Following G protein dissociation, the GPCR is phosphorylated by G protein receptor kinases (GRK) at the intracellular loops and carboxy-terminal tail. The phosphorylated GPCR increases its affinity for interacting with arrestin proteins. Arrestins and GRK compete for the same internal site of the GPCR where they interact with the G protein and thus promote GPCR desensitization. Recruited arrestins further promote GPCR internalization by interacting with AP2, clathrin and dynamin. Arrestins can also serve as scaffolds that allow the assembly of signaling complexes, including for kinase enzymes.

When GTP is hydrolyzed to GDP, the α, β and γ subunits re-associate to form a G protein. The G protein then binds a GPCR.

A general approach for detecting protein-protein interactions relies on protein-fragment complementation. Protein-fragment complementation methods involve the fusion of complementary fragments of a reporter protein to two proteins of interest. When the two proteins of interest interact, the complementary fragments of the reporter protein are brought into sufficiently close proximity that they spontaneously re-fold and can generate a detectable signal.

In view of the above, the present disclosure seeks to provide a real-time assay of simultaneous recruitment of arrestin isoforms, such as β-arrestin isoforms, to a receptor. This and other objects and advantages, as well as inventive features, will be apparent from the description provided herein.

### SUMMARY

A method of determining if a test agent preferentially recruits an isoform of an arrestin, such as an isoform of β-arrestin, to a receptor is provided. The method comprises (i) contacting a cell, which expresses (a) a G protein-coupled receptor (GPCR) linked to a fragment of an enzyme, which, in the presence of a substrate for the enzyme, produces an optically detectable signal upon refolding with a complementary fragment of the enzyme linked to an isoform of β-arrestin, (b) a first isoform of β-arrestin linked to a first complementary fragment of the enzyme, which produces an optically detectable signal of a first λₘₐₓ (i.e., peak emission wavelength) upon refolding with the complementary fragment of the enzyme linked to the GPCR and, (c) a second isoform of β-arrestin linked to a second complementary fragment of the enzyme, which produces an optically detectable signal of a second λₘₐₓ upon refolding with the complementary fragment of the enzyme linked to the GPCR, with (a′) a substrate for the enzyme, and (b′) the test agent; and (ii) detecting, and optionally quantitating, the optically detectable signal of the first λₘₐₓ and the optically detectable signal of the second λₘₐₓ, wherein an absolute value of a ratio of the amount of signal of the first λₘₐₓ to the amount of signal of the second λₘₐₓ is compared to an absolute value of a corresponding ratio obtained for a reference agent and indicates whether the agent preferentially recruits an isoform of β-arrestin to the receptor. The method also can be used to evaluate kinetic differences in arrestin recruitment.

The test agent can be an agonist, an antagonist, a sensitizing agent, or a desensitizing agent. Any suitable reference agent can be used. The enzyme can be a luciferase, such as a click beetle green (CBG) luciferase, in which case the substrate for the enzyme can be D-luciferin. The fragment of the enzyme linked to the GPCR can be a C-terminal fragment. The first complementary fragment of the enzyme linked to the first isoform of an arrestin, such as a first isoform of β-arrestin, and the second complementary fragment of the enzyme linked to the second isoform of an arrestin, such as a second isoform of β-arrestin, can be N-terminal fragments. The first complementary fragment of the enzyme linked to the first isoform of an arrestin, such as a first isoform of β-arrestin, can be an N-terminal fragment from a CBG luciferase. The second complementary fragment of the enzyme linked to the second isoform of an arrestin, such as a second isoform of β-arrestin, can be an N-terminal fragment from a click beetle red (CBR) luciferase. The GPCR can be the δ opioid receptor (DOR). The first isoform of β-arrestin can be β-arrestin 1 (Barr1). The second isoform of β-arrestin can be β-arrestin 2 (Barr2). The cell can be in a well on a microtiter plate. The cell can be in a living animal. A cell lysate preparation can be used in place of the cell.

A biosensor is also provided. The biosensor comprises (i) (a) a cell, which expresses a GPCR linked to a fragment of an enzyme, which, in the presence of a substrate for the enzyme, produces an optically detectable signal upon refolding with a complementary fragment of the enzyme linked to an isoform of an arrestin, such as β-arrestin, or (b) a cell lysate prepared from (a), and (ii) (a) a first isoform of an arrestin, such as a β-arrestin, linked to a first complementary fragment of the enzyme, which produces an optically detectable signal of a first λₘₐₓ upon refolding with the complementary fragment of the enzyme linked to the GPCR, and (b) a second isoform of an arrestin, such as a β-arrestin, linked to a second complementary fragment of the enzyme, which produces an optically detectable signal of a second λₘₐₓ upon refolding with the complementary fragment of the enzyme linked to the GPCR.

The biosensor can further comprise (iii) a substrate for the enzyme. The enzyme can be luciferase, such as a click beetle luciferase, in which case the substrate for the enzyme can be D-luciferin. The fragment of the enzyme linked to the GPCR can be a C-terminal fragment. The C-terminal fragment can be a C-terminal fragment of CBG luciferase. The first complementary fragment of the enzyme linked to the first isoform of an arrestin, such as a first isoform of β-arrestin, and the second complementary fragment of the enzyme linked to the second isoform of an arrestin, such as a second isoform of β-arrestin, can be N-terminal fragments. The first complementary fragment of the enzyme linked to the first isoform of an arrestin, such as a first isoform of β-arrestin, can be an N-terminal fragment from a CBG luciferase. The second complementary fragment of the enzyme linked to the second isoform of an arrestin, such as a second isoform of β-arrestin, can be an N-terminal fragment from a CBR luciferase. The GPCR can be the DOR. The first isoform of an arrestin, such as a first isoform of β-arrestin, can be β-arrestin 1 (Barr1). The second isoform of an arrestin, such as a second isoform of β-arrestin, can be β-arrestin 2 (Barr2). The cell or cell lysate can be in a well on a substrate, such as a microtiter plate, e.g., a 96-well microtiter plate, or similar format.

A bioarray is further provided. The bioarray comprises the biosensors.

A kit is still further provided. The kit comprises (a) a biosensor or a bioarray and (b) instructions for determining if an agent preferentially recruits an isoform of an arrestin, such as an isoform of β-arrestin, to a GPCR. The kit also can be used to evaluate kinetic differences in arrestin recruitment.

### BRIEF DESCRIPTION OF THE FIGURES

FIG. 1 is a schematic diagram of the method.

FIG. 2 is an example of dose-response curves (graph of log10 drug vs. construct signal) in 508 (CBGnt-Barr1) and 620 (CBRnt-Barr2) nm channels obtained with two δ opioid receptor (DOR) agonists (Leu-Enk [leucine-enkephalin; YGGFL [SEQ ID NO: 15] and SNC80 [(+)-4-[(αR)-α-((2S,5R)-4-Allyl-2,5-dimethyl-1-piperazinyl)-3-methoxybenzyl]-N,N-diethylbenzamide] in cells triply transfected with a DOR-CBGct, a CBGnt-Barr1, and a CBRnt-Barr2 construct. Half maximal effect concentration (EC50) values are included in a table under the graph.

FIG. 3A is a heat map showing the fold change in signal in response to Leu-enkephalin, the endogenous agonist for DOR, for cells transfected with the indicated DOR-CBGct and CBGnt-Barr1 constructs.

FIG. 3B is a heat map showing the fold change in signal in response to Leu-enkephalin, the endogenous agonist for DOR, for cells transfected with the indicated DOR-CBGct and CBRnt-Barr2 constructs.

FIG. 4A is a graph of time post-leu-enkephalin (m) vs. 508 nm (Barr1) luminescence (RLU) in cells triply transfected with a DOR-CBGct, a CBGnt-Barr1, and a CBRnt-Barr2 construct.

FIG. 4B is a graph of time post-leu-enkephalin (m) vs. 620 nm (Barr2) luminescence (RLU) in cells triply transfected with a DOR-CBGct, a CBGnt-Barr1, and a CBRnt-Barr2 construct.

FIG. 4C is a graph of log10 [leu-enkephalin (M)] vs. fold change Barr1 (508 nm) signal at a single time point in FIG. 4A.

FIG. 4D is a graph of time post-leu-enkephalin (m) vs. fold change response to leu-enkephalin in the cells in FIGS. 4A and 4B.

FIG. 4E is a graph of time post-leu-enkephalin (m) vs. measured log10 EC50 to leu-enkephalin in the cells in FIGS. 4A and 4B.

FIG. 5A is a graph of log10 [drug (M)] vs. mean normalized 508 nm signal (Barr1) for a panel of DOR agonists in cells triply transfected with a DOR-CBGct, a CBGnt-Barr1, and a CBRnt-Barr2 construct.

FIG. 5B is a graph of log10 [drug (M)] vs. mean normalized 620 nm signal (Barr2) for a panel of DOR agonists in cells triply transfected with a DOR-CBGct, a CBGnt-Barr1, and a CBRnt-Barr2 construct.

FIG. 5C is a graph of drug vs. Δ% efficacy Barr1-Barr2 corresponding to curves in FIGS. 5A and 5B. ***, p<0.001 with respect to Leu-Enk reference.

FIG. 5D is a graph of drug or standard (Leu-Enk) vs. Δlog10 EC50 Barr1-Barr2 corresponding to curves in FIGS. 5A and 5B.

FIG. 6A is a graph of time (m) vs. normalized β-arrestin signal for two DOR agonists in cells triply transfected with a DOR-CBGct, a CBGnt-Barr1, and a CBRnt-Barr2 construct.

FIG. 6B is a graph of time (m) vs. normalized β-arrestin signal, which is an enlargement of the portion of the graph outlined in FIG. 6A.

FIG. 7A is a graph of log10 [angiotensin II, M] vs. fold change in angiotensin type 1 receptor (AGTR1):β-arrestin signal in cells triply transfected with an AGTR1-CBGct, a CBGnt-Barr1, and a CBRnt-Barr2 construct.

FIG. 7B is a graph of log10 [UNC9994, M] vs. fold change in dopamine D2 receptor (D2R):β-arrestin signal in cells triply transfected with a D2R-CBGct, a CBGnt-Barr1, and a CBRnt-Barr2 construct.

FIG. 7C is a graph of log10 [dynorphin17, M] vs. fold change in kappa opioid receptor:β-arrestin signal in cells triply transfected with a kappa opioid receptor-CBGct, a CBGnt-Barr1, and a CBRnt-Barr2 construct.

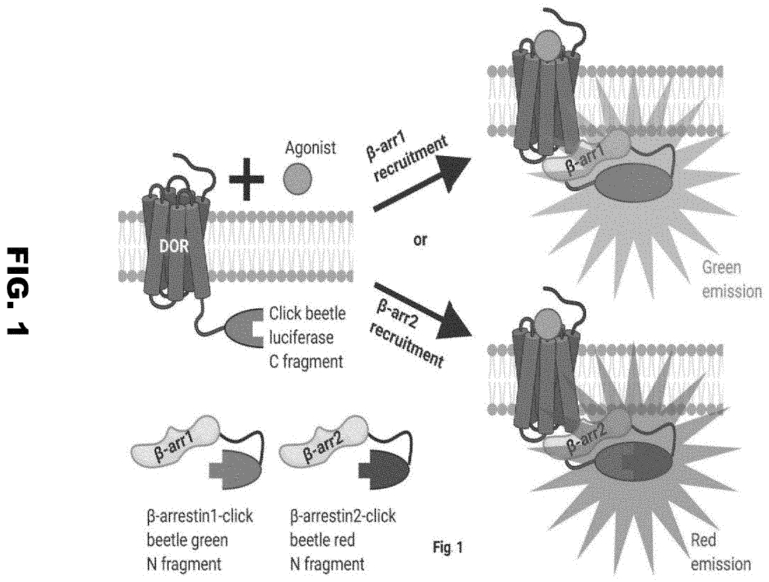

### DETAILED DESCRIPTION

The present disclosure is directed to the development of a simultaneous, real-time assay that measures the recruitment of two arrestin isoforms, such as two β-arrestin isoforms, to G protein-coupled receptors (GPCRs). The use of a single cell (or a cell lysate preparation) system employing a single substrate enables the identification of arrestin isoform bias, such as β-arrestin isoform bias, for an agent, such as an agonist, an antagonist, a sensitizing agent, or a desensitizing agent, and enables the measurement of recruitment kinetics.

In view of the above, a method of determining if a test agent preferentially recruits an isoform of arrestin, such as an isoform of β-arrestin, to a G protein-coupled receptor (GPCR) is provided. The method comprises (i) contacting a cell, which expresses (a) a GPCR linked to a fragment of an enzyme, which, in the presence of a substrate for the enzyme, produces an optically detectable signal upon refolding with a complementary fragment of the enzyme linked to an isoform of β-arrestin, (b) a first isoform of β-arrestin linked to a first complementary fragment of the enzyme, which produces an optically detectable signal of a first λₘₐₓ (i.e., peak emission wavelength) upon refolding with the complementary fragment of the enzyme linked to the GPCR and, (c) a second isoform of β-arrestin linked to a second complementary fragment of the enzyme, which produces an optically detectable signal of a second λₘₐₓ upon refolding with the complementary fragment of the enzyme linked to the GPCR, with (a′) a substrate for the enzyme, and (b′) the test agent; and (ii) detecting, and optionally quantitating, the optically detectable signal of the first λₘₐₓ and the optically detectable signal of the second λₘₐₓ, wherein an absolute value of a ratio of the amount of signal of the first λₘₐₓ to the amount of signal of the second λₘₐₓ is compared to an absolute value of a corresponding ratio obtained for a reference agent and indicates whether the agent preferentially recruits an isoform of β-arrestin to the receptor. The method also can be used to evaluate kinetic differences in arrestin recruitment.

The test agent can be an agonist, an antagonist, a sensitizing agent, or a desensitizing agent. Any suitable reference agent can be used. The cell can be in a well on a microtiter plate. The cell can be in a living animal. A cell lysate preparation can be used in place of the cell.

While the disclosure is illustrated with the delta opioid receptor (DOR), the method can be used to measure recruitment of arrestin isoforms to other GPCRs, such as other opioid receptors (e.g., the κ opioid receptor (KOR) or the μ opioid receptor (MOR1)), adrenergic receptors (e.g., α1B, α2A, α2B, α2C, β1, and β2), dopamine receptors (e.g., D1A, D1B, D2, D3, and D4), muscarinic receptors (e.g., m1, m2, m3, and m4), serotonergic receptors (e.g., HTR, 5HT1A, and 5HT2A), chemokine receptors (e.g., CXCR2, CXCR4, and CCR5), EDG receptors (e.g., Edg1, Edg2, Edg3, and Edg5), GI receptors (e.g., CCK-A, CCK-B, glucagon, secretin, and VIP), G protein-coupled estrogen receptor (GPER), prostaglandin receptors (e.g., EP3 and EP4), f-met-leu-pro receptor (FMLP), angiotensin type 1 and 2 receptors (AGTR1 and AGTR2), corticotropin releasing factor receptor (CRFR), ETAR (endothelin A receptor), NK-1 (neurotensin receptor), octopamine receptor (OAMB), oxytocin receptor (OXTR), thyroid releasing hormone receptor (TRHR), rhodopsin, neuromedin B receptor (NMBR), adrenoceptor α1A (ADRA1A), motilin receptor (MLNR), vasopressin receptor 1β (AVPR1B), and V2 (vasopressin receptor). The method also can be used to measure recruitment of isoforms of GPCR kinases (GRKs); there are six GRKs.

Any suitable cells can be used. Examples include eukaryotic cells, such as mammalian cells, such as human cells. The cells may naturally express the GPCR or may be engineered to express the GPCR. Desirably, the GPCR is a functional receptor in the cell membrane of the cell.

Protein-protein interactions are monitored in living cells (or cell lysate preparations, or small, living animals) based on complementation of a split enzyme, such as ubiquitin, β-galactosidase, β-lactamase, dihydrofolate reductase, green fluorescent protein, firefly luciferase (e.g., P. pyralis), Renilla luciferase, Gaussia luciferase, Oplophorus luciferase, and click beetle (Pyrophorus plagiothalamus) luciferase. The split reporter proteins may differ in cognate substrate, spectral properties, and quality of emission.

The use of a split luciferase can be advantageous. The use of split luciferases is advantageous for imaging the interaction of two proteins of interest in an intact cell. Tissue attenuation of emitted photons also affords imaging when applied directly to small living animals. And, given that the luciferases emit light as a result of a chemical reaction, they do not require a high-energy source of excitation, which can lead to background noise and tissue damage, both of which can affect signal measurement.

The use of a split luciferase from click beetle offers other advantages and is exemplified herein. The light emitted is very bright; the photon count is estimated to be over 10-fold higher than that of firefly luciferase. And the spectrum of light emitted by click beetle luciferase is pH-independent, whereas that of firefly luciferase is not. Click beetle luciferase also shows little color sensitivity to temperature.

The enzyme can be split into amino-terminal (N-terminal) and carboxy-terminal (C-terminal) fragments. Any suitable amino-terminal and carboxy-terminal fragments can be used. Such fragments can be identified using semi-rational library screening, for example. The pair of fragments desirably are highly sensitive and provide a high signal-to-background ratio.

Arrestin isoforms, such as β-arrestin isoforms, interact with GPCRs on the plasma membrane. When a ligand binds to a GPCR, it triggers phosphorylation of the GPCR. The phosphorylated GPCR then interacts with a β-arrestin. The interaction between the GPCR and β-arrestin brings the fragment of the reporter protein attached to the GPCR in close proximity with the complementary fragment of the reporter protein attached to the β-arrestin. The complementary fragments of the reporter protein, e.g., a split luciferase from click beetle, spontaneously re-fold and can generate a detectable signal upon chemical reaction with a substrate (e.g., D-luciferin, which generates light in the presence of ATP and Mg2+; ATP is generated naturally in any suitable cell, such as a mammalian cell, such as a human cell; Mg2+ is included in standard cell culture media needed to keep any suitable cell alive).

“Arrestin” means all types of naturally occurring and engineered variants of arrestin including, but not limited to, arrestin 1 (visual arrestin), arrestin 2 (β-arrestin 1 or Barr1), arrestin 3 (β-arrestin 2 or Barr2), and arrestin 4.

One fragment of a reporter protein, such as a C-terminal fragment (e.g., a C-terminal fragment of a luciferase, such as a C-terminal fragment of a click beetle luciferase, such as click beetle green (CBG) luciferase), is attached to a receptor, e.g., a GPCR, such as a DOR. The complementary fragment of the reporter protein, such as an N-terminal fragment (e.g., an N-terminal fragment of a luciferase, such as an N-terminal fragment of a click beetle luciferase), is attached to an isoform of an arrestin (e.g., an isoform of a β-arrestin). When a fragment (e.g., an N-terminal fragment) of a first reporter protein, such as a green luciferase (e.g., a CBG luciferase), is attached to a first isoform of arrestin and binds to a receptor (e.g., a GPCR, such as a DOR) in the presence of substrate (e.g., D-luciferin), such that a light of a first color (e.g., green, such as when λₘₐₓ is within 500 nm to 550 nm) is generated, and a fragment (e.g., an N-terminal fragment) of a second reporter protein, such as a red luciferase (e.g., a click beetle red (CBR) luciferase), is attached to a second isoform of arrestin and binds to a receptor (e.g., a GPCR, such as a DOR) in the presence of substrate (e.g., D-luciferin), such that a light of a second color (e.g., orange to red, such as when λₘₐₓ is within 600 nm to 700 nm) is generated, the recruitment of different isoforms of an arrestin (such as different isoforms of β-arrestin) to a receptor, such as a GPCR (e.g., DOR), can be assayed in real-time.

The terminus of the reporter protein fragment, such as that of a luciferase, e.g., a click beetle luciferase, and the linker length are important factors for optimizing the assay. For example, the major substrate reaction center is located in the N-terminus of a luciferase, such as a click beetle luciferase. Linker length can be optimized in accordance with methods known in the art and exemplified herein. An N-terminal fragment, such as a fragment comprising residues 1-415, can be optimal for click beetle green luciferase, such as in a construct comprising an N-terminal fragment of CBG and Barr1. An N-terminal fragment, such as a fragment comprising residues 1-413, can be optimal for click beetle red luciferase, such as in a construct comprising an N-terminal fragment of CBR and Barr2. Optimal N-terminal fragments of click beetle luciferase can comprise residues 1 through the C-terminus of the fragment, which can be 406-417 (Misawa et al., Anal Chem 82: 2552-2560 (2010)), whereas optimal N-terminal fragments of firefly luciferase can comprise residues 398-416 (Luker et al., PNAS 101(33): 12288-12293 (2004)). A suitable complementary C-terminal fragment, such as a fragment comprising residues 395-542, can be optimal for click beetle green luciferase, such as in a construct comprising a C-terminal fragment of CBG and DOR. Optimal C-terminal fragments of click beetle luciferase can run through the C-terminus of the full length protein (residue 542) and begin at residues 389-413 (Misawa et al. (2010), supra), whereas optimal C-terminal fragments of firefly luciferase can continue through the C-terminus of the full-length protein (residue 550) and begin at residues 388-408 (Luker et al. (2004), supra). Suitable complementary pairs of fragments for click beetle luciferase are exemplified herein but are not limited to those exemplified. Suitable complementary pairs of fragments for firefly luciferase include, but are not limited to, an N-terminal click beetle green luciferase fragment comprising residues 1-415 or an N-terminal click beetle red luciferase fragment comprising residues 1-413 and a C-terminal fragment comprising residues 395-542. If desired, an N-terminal fragment of one enzyme can be used in combination with a C-terminal fragment of another enzyme.

The method includes providing at least one cell (or cell lysate preparation, or small, living animal) that expresses a receptor, such as a GPCR (e.g., a DOR) and a plurality (e.g., at least two) of isoforms of an arrestin (e.g., a plurality of isoforms of β-arrestin), wherein each isoform is conjugated to a fragment, such as an N-terminal fragment, of a different reporter protein. The plurality of isoforms of the arrestin (e.g., the plurality of isoforms of β-arrestin), is substantially evenly distributed in the cytoplasm of at least one cell (or in the cell lysate preparation, or small, living animal).

A substrate, such as a microtiter plate, e.g., a 96-well microtiter plate, or a similar format, can be used for high-throughput, rapid and sensitive screening of selective arrestin isoform recruitment in the presence of multiple (e.g., hundreds or thousands) agents, such as agonists, antagonists, sensitizing agents, and desensitizing agents. Arrestin-receptor interactions can be analyzed spatially and temporally in individual living cells, such as in living cells individually placed in separate wells on a substrate, such as a microtiter plate, e.g., a 96-well microtiter plate, or similar format. Alternatively, cell lysates can be used to measure total cell arrestin-receptor interactions in the cell (e.g., Jones et al., J Biol Chem 296(100133): (2021)). The readout signal can be detected with high sensitivity, accuracy, and reproducibility. If desired, arrestin-receptor interactions can be analyzed in small, living animals.

The cellular distribution of signal-generating, receptor-bound isoforms of an arrestin (e.g., isoforms of β-arrestin) can be visualized using any suitable method known in the art, e.g., flow cytometry, bioluminescence microscopy, or fluorescence confocal microscopy. A computer can analyze an image of the cellular distribution, and the distribution can be quantified.

In view of the above, a biosensor is also provided. The biosensor comprises (i) (a) a cell, which expresses a GPCR linked to a fragment of an enzyme, which, in the presence of a substrate for the enzyme, produces an optically detectable signal upon refolding with a complementary fragment of the enzyme linked to an isoform of β-arrestin, or (b) a cell lysate prepared from (a), and(ii) (a) a first isoform of an arrestin, such as a β-arrestin, linked to a first complementary fragment of the enzyme, which produces an optically detectable signal of a first λₘₐₓ upon refolding with the complementary fragment of the enzyme linked to the GPCR, and (b) a second isoform of an arrestin, such as a β-arrestin, linked to a second complementary fragment of the enzyme, which produces an optically detectable signal of a second λₘₐₓ upon refolding with the complementary fragment of the enzyme linked to the GPCR.

The biosensor can further comprise (iii) a substrate for the enzyme. The enzyme can be luciferase, such as a click beetle luciferase, in which case the substrate for the enzyme can be D-luciferin, which generates light in the presence of ATP and Mg2+ (ATP is generated naturally in any suitable cell, such as a mammalian cell, such as a human cell; Mg2+ is included in standard cell culture media needed to keep any suitable cell alive). The fragment of the enzyme linked to the GPCR can be a C-terminal fragment. The C-terminal fragment can be a C-terminal fragment of CBG luciferase. The first complementary fragment of the enzyme linked to the first isoform of an arrestin, such as a first isoform of β-arrestin, and the second complementary fragment of the enzyme linked to the second isoform of an arrestin, such as a second isoform of β-arrestin, can be N-terminal fragments. The first complementary fragment of the enzyme linked to the first isoform of an arrestin, such as the first isoform of β-arrestin, can be an N-terminal fragment from a CBG luciferase. The second complementary fragment of the enzyme linked to the second isoform of an arrestin, such as the second isoform of β-arrestin, can be an N-terminal fragment from a CBR luciferase. The GPCR can be the DOR. The first isoform of β-arrestin can be β-arrestin 1 (Barr1). The second isoform of β-arrestin can be β-arrestin 2 (Barr2). The cell or cell lysate can be in a well on a substrate, such as a microtiter plate, e.g., a 96-well microtiter plate, or similar format.

Also in view of the above, a bioarray is provided. The bioarray comprises at least one biosensor, such as at least one single cell biosensor, or at least one cell lysate biosensor. Desirably, the bioarray comprises a multitude of biosensors, such as a multitude of single cell biosensors, or a multitude of cell lysate biosensors. The multitude can range from at least 2 to hundreds and even thousands.

Also in view of the above, a kit is provided. The kit comprises (a) a biosensor or a bioarray and (b) instructions for determining if an agent preferentially recruits an isoform of β-arrestin to a GPCR. The kit also can be used to evaluate kinetic differences in arrestin recruitment.

If desired, the method can be performed in a living animal, such as a small living animal, e.g., a mammal, such as a mouse. When the method is performed in a living animal, whole-body imaging can be carried out.

### EXAMPLES

The following examples serve to illustrate the present disclosure. The examples are not intended to limit the scope of the claimed invention in any way.

### Example 1

### Cloning

A pCDNA3.1 vector containing a mouse (Mus musculus) delta opioid receptor (DOR) gene N-terminally tagged with an influenza signal peptide for expression and a FLAG tag for future labeling studies (Evans et al., Science 258: 1952-1955 (1992)) was digested with NotI and PasI. A NotI-PasI-digested fragment of a vector containing the C-terminal (ct; residues 395-542) click beetle green luciferase (CBG) fragment (CBGct) was ligated into the pCDNA3.1 vector (Wood et al., Science 244: 700-702 (1989)).

A pCDNA3.1 vector encoding Barr1 (β-arrestin 1; cDNA.org) was digested with NheI and BspEI. A NheI-BspEI-digested fragment of a vector containing the N-terminal (nt; residues 1-413) CBG fragment (CBGnt) was ligated into the pCDNA3.1 vector.

A pCDNA3.1 vector encoding Barr2 (β-arrestin 2; cDNA.org) was digested with NheI and AgeI. A NheI-AgeI-digested fragment of a vector containing the N-terminal (nt; residues 1-413) click beetle red (CBR) luciferase fragment (CBRnt) was ligated into the pCDNA3.1 vector.

All ligations were performed with T4 DNA ligase (New England Biolabs (Ipswich, Mass.)) according to the manufacturer's guidelines. For each construct, the portion of DOR, Barr1, or Barr2 encoded was set to replace the original sequence excised by the restriction enzymes without altering the protein's sequence in the final construct. The exception was the removal of the start codon methionine in the arrestin constructs because it was no longer necessary once the N-terminal luciferase fragments were added.

CBGct ligation fragment: |NotI|-DOR (338-372)-Gly-Ser-CBGct (395-542)-STOP-|PasI|:

[SEQ ID NO: 12]
GCGGCCGCCAAGAACCCGGCAGTCTCCGTCGTCCCCGCCAGGCCACCACG
CGTGAGCGTGTCACTGCCTGCACCCCCTCCGACGGCCCGGGCGGTGGCGC
CGCCGCCGGCAGTAGCAAGGGTTATGTCAATAACGTTGAAGCTACCAAGG
AGGCCATCGACGACGACGGCTGGTTGCATTCTGGTGATTTTGGATATTAC
GACGAAGATGAGCATTTTTACGTCGTGGATCGTTACAAGGAGCTGATCAA
ATACAAGGGTAGCCAGGTTGCTCCAGCTGAGTTGGAGGAGATTCTGTTGA
AAAATCCATGCATTCGCGATGTCGCTGTGGTCGGCATTCCTGATCTGGAG
GCCGGCGAACTGCCTTCTGCTTTCGTTGTCAAGCAGCCTGGTAAAGAAAT
TACCGCCAAAGAAGTGTATGATTACCTGGCTGAACGTGTGAGCCATACTA
AGTACTTGCGTGGCGGCGTGCGTTTTGTTGACTCCATCCCTCGTAACGTA
ACAGGCAAAATTACCCGCAAGGAGCTGTTGAAACAATTGTTGGAGAAGGC
CGGCGGTTGACCCTGGG,

which encodes the amino acid sequence:

### |NotI|-DOR (338-372)-Gly-Ser-CBGct (395-542)-STOP-|PasI|:

[SEQ ID NO: 22]
|NotI|-GRQEPGSLRRPRQATTRERVTACTPSDGPGGGAAA-GS-
SKGYVNNVEATKEAIDDDGWLHSGDFGYYDEDEHFYVVDRYKELIKYKGS
QVAPAELEEILLKNPCIRDVAVVGIPDLEAGELPSAFVVKQPGKEITAKE
VYDYLAERVSHTKYLRGGVRFVDSIPRNVTGKITRKELLKQLLEKAGG-
STOP-|PasI|,

where |NotI| and |PasI| refer to the extra nucleotides outside the coding region for the amino acid sequence listed that encode the restriction enzyme digestion sites.

CBGnt ligation fragment: |NheI|-CBGnt (1-413)-Ser-Gly-Leu-Lys-Ser-Arg-Arg-Ala-Leu-Asp-Ser-Ala-Barr1 (2-169)-|BspEI|:

[SEQ ID NO: 13]
GCTAGCgccgccAccATGGTGAAGCGTGAGAAAAATGTCATCTATGGCCC
TGAGCCTCTCCATCCTTTGGAGGATTTGACTGCCGGCGAAATGCTGTTTC
GTGCTCTCCGCAAGCACTCTCATTTGCCTCAAGCCTTGGTCGATGTGGTC
GGCGATGAATCTTTGAGCTACAAGGAGTTTTTTGAGGCAACCGTCTTGCT
GGCTCAGTCCCTCCACAATTGTGGCTACAAGATGAACGACGTCGTTAGTA
TCTGTGCTGAAAACAATACCCGTTTCTTCATTCCAGTCATCGCCGCATGG
TATATCGGTATGATCGTGGCTCCAGTCAACGAGAGCTACATTCCCGACGA
ACTGTGTAAAGTCATGGGTATCTCTAAGCCACAGATTGTCTTCACCACTA
AGAATATTCTGAACAAAGTCCTGGAAGTCCAAAGCCGCACCAACTTTATT
AAGCGTATCATCATCTTGGACACTGTGGAGAATATTCACGGTTGCGAATC
TTTGCCTAATTTCATCTCTCGCTATTCAGACGGCAACATCGCAAACTTTA
AACCACTCCACTTCGACCCTGTGGAACAAGTTGCAGCCATTCTGTGTAGC
AGCGGTACTACTGGACTCCCAAAGGGAGTCATGCAGACCCATCAAAACAT
TTGCGTGCGTCTGATCCATGCTCTCGATCCACGCGTGGGCACTCAGCTGA
TTCCTGGTGTCACCGTCTTGGTCTACTTGCCTTTCTTCCATGCTTTCGGC
TTTAGCATTACTTTGGGTTACTTTATGGTCGGTCTCCGCGTGATTATGTT
CCGCCGTTTTGATCAGGAGGCTTTCTTGAAAGCCATCCAAGATTATGAAG
TCCGCAGTGTCATCAACGTGCCTAGCGTGATCCTGTTTTTGTCTAAGAGC
CCACTCGTGGACAAGTACGACTTGTCTTCACTGCGTGAATTGTGTTGCGG
TGCCGCTCCACTGGCTAAGGAGGTCGCTGAAGTGGCCGCCAAACGCTTGA
ATCTTCCAGGGATTCGTTGTGGCTTCGGCCTCACCGAATCTACCAGCGCA
AACATTCACTCTCTCGGGGATGAGTTTAAGAGCGGCTCTTTGGGCCGTGT
CACTCCACTCATGGCTGCTAAGATCGCTGATCGCGAAACTGGTAAGGCTT
TGGGCCCGAACCAAGTGGGCGAGCTGTGTATCAAAGGCCCTATGGTGAGC
AAGGGTTATGTCAATAACGTTGAAGCTACCAAGGAGGCCATCGACGACGA
CGGCAGCGGCCTGAAGAGCAGGAGGGCCCTGGACAGCGCTGGCGACAAAG
GGACCCGAGTGTTCAAGAAGGCCAGTCCAAATGGAAAGCTCACCGTCTAC
CTGGGAAAGCGGGACTTTGTGGACCACATCGACCTCGTGGACCCTGTGGA
TGGTGTGGTCCTGGTCGATCCTGAGTATCTCAAAGAGCGGAGAGTCTATG
TGACGCTGACCTGCGCCTTCCGCTATGGCCGGGAGGACCTGGATGTCCTG
GGCCTGACCTTTCGCAAGGACCTGTTTGTGGCCAACGTACAGTCGTTCCC
ACCGGCCCCCGAGGACAAGAAGCCCCTGACGCGGCTGCAGGAACGCCTCA
TCAAGAAGCTGGGCGAGCACGCTTACCCTTTCACCTTTGAGATCCCTCCA
AACCTTCCATGTTCTGTGACACTGCAGCCGGGGCCCGAAGACACGGGGAA
GGCTTGCGGTGTGGACTATGAAGTCAAAGCCTTCTGCGCGGAGAATTTGG
AGGAGAAGATCCACAAGCGCAATTCTGTGCGTCTGGTCATCCGGA,

which encodes the amino acid sequence:

### |NheI|-CBGnt (1-413)- Ser-Gly-Leu-Lys-Ser-Arg-Arg-Ala-Leu-Asp-Ser-Ala-Barr1 (2-169)-|BspEI|:|NheI|-

[SEQ ID NO: 23]
MVKREKNVIYGPEPLEEPLEDLTAGEMLFRALRKHSIALPQALVDVVGDE
SLSYKEFFEATVLLAQSLHNCGYKMNDVVSICAENNTRFFIPVIAAWYIG
MIVAPVNESYIPDELCKVMGISKPQIVFTTKNILNKVLEVQSRTNFIKRI
IILDTVENIFIGCESLPNFISRYSDGNIANFKPLEEFDPVEQVAAILCSS
GTTGLPKGVMQTHQNICVRLITIALDPRVGTQLIPGVTVLVYLPFFHAFG
FSITLGYFMVGLRVIMFRRFDQEAFLKAIQDYEVRSVINVPSVILFLSKS
PLVDKYDLSSLRELCCGAAPLAKEVAEVAAKRLNLPGIRCGFGLTESTSA
NIFISLGDEFKSGSLGRVTPLMAAKIADRETGKALGPNQVGELOKGPMVS
KGYVNNVEATKEAIDDDG-SGLKSRRALDSA-
GDKGTRVFKKASPNGKLTVYLGKRDFVDHIDLVDPVDGVVLVDPEYLKER
RVYVTLTCAFRYGREDLDVLGLTFRKDLFVANVQSFPPAPEDKKPLTRLQ
ERLIKKLGEHAYPFTFEIPPNLPCSVTLQPGPEDTGKACGVDYEVKAFCA
ENLEEKIHKRNSVRLVIR-|BspEI|,

where |NheI| and |BspEI| refer to the extra nucleotides outside the coding region for the amino acid sequence listed that encode the restriction enzyme digestion sites.

CBRnt ligation fragment: |NheI|-CBRnt (1-413)-Ser-Gly-Leu-Lys-Ser-Arg-Arg-Ala-Leu-Asp-Ser-Ala-Barr2 (2-196)-|AgeI|:

[SEQ ID NO: 14]
GCTAGCgccgccAccATGGTAAAGCGTGAGAAAAATGTCATCTATGGCCC
TGAGCCTCTCCATCCTTTGGAGGATTTGACTGCCGGCGAAATGCTGTTTC
GTGCTCTCCGCAAGCACTCTCATTTGCCTCAAGCCTTGGTCGATGTGGTC
GGCGATGAATCTTTGAGCTACAAGGAGTTTTTTGAGGCAACCGTCTTGCT
GGCTCAGTCCCTCCACAATTGTGGCTACAAGATGAACGACGTCGTTAGTA
TCTGTGCTGAAAACAATACCCGTTTCTTCATTCCAGTCATCGCCGCATGG
TATATCGGTATGATCGTGGCTCCAGTCAACGAGAGCTACATTCCCGACGA
ACTGTGTAAAGTCATGGGTATCTCTAAGCCACAGATTGTCTTCACCACTA
AGAATATTCTGAACAAAGTCCTGGAAGTCCAAAGCCGCACCAACTTTATT
AAGCGTATCATCATCTTGGACACTGTGGAGAATATTCACGGTTGCGAATC
TTTGCCTAATTTCATCTCTCGCTATTCAGACGGCAACATCGCAAACTTTA
AACCACTCCACTTCGACCCTGTGGAACAAGTTGCAGCCATTCTGTGTAGC
AGCGGTACTACTGGACTCCCAAAGGGAGTCATGCAGACCCATCAAAACAT
TTGCGTGCGTCTGATCCATGCTCTCGATCCACGCTACGGCACTCAGCTGA
TTCCTGGTGTCACCGTCTTGGTCTACTTGCCTTTCTTCCATGCTTTCGGC
TTTCATATTACTTTGGGTTACTTTATGGTCGGTCTCCGCGTGATTATGTT
CCGCCGTTTTGATCAGGAGGCTTTCTTGAAAGCCATCCAAGATTATGAAG
TCCGCAGTGTCATCAACGTGCCTAGCGTGATCCTGTTTTTGTCTAAGAGC
CCACTCGTGGACAAGTACGACTTGTCTTCACTGCGTGAATTGTGTTGCGG
TGCCGCTCCACTGGCTAAGGAGGTCGCTGAAGTGGCCGCCAAACGCTTGA
ATCTTCCAGGGATTCGTTGTGGCTTCGGCCTCACCGAATCTACCAGTGCG
ATTATCCAGACTCTCGGGGATGAGTTTAAGAGCGGCTCTTTGGGCCGTGT
CACTCCACTCATGGCTGCTAAGATCGCTGATCGCGAAACTGGTAAGGCTT
TGGGCCCGAACCAAGTGGGCGAGCTGTGTATCAAAGGCCCTATGGTGAGC
AAGGGTTATGTCAATAACGTTGAAGCTACCAAGGAGGCCATCGACGACGA
CGGCAGCGGCCTGAAGAGCAGGAGGGCCCTGGACAGCGCTGGGGAGAAAC
CCGGGACCAGGGTCTTCAAGAAGTCGAGCCCTAACTGCAAGCTCACCGTG
TACTTGGGCAAGCGGGACTTCGTAGATCACCTGGACAAAGTGGACCCTGT
AGATGGCGTGGTGCTTGTGGACCCTGACTACCTGAAGGACCGCAAAGTGT
TTGTGACCCTCACCTGCGCCTTCCGCTATGGCCGTGAAGACCTGGATGTG
CTGGGCTTGTCCTTCCGCAAAGACCTGTTCATCGCCACCTACCAGGCCTT
CCCCCCGGTGCCCAACCCACCCCGGCCCCCCACCCGCCTGCAGGACCGGC
TGCTGAGGAAGCTGGGCCAGCATGCCCACCCCTTCTTCTTCACCATACCC
CAGAATCTTCCATGCTCCGTCACACTGCAGCCAGGCCCAGAGGATACAGG
AAAGGCCTGCGGCGTAGACTTTGAGATTCGAGCCTTCTGTGCTAAATCAC
TAGAAGAGAAAAGCCACAAAAGGAACTCTGTGCGGCTGGTGATCCGAAAG
GTGCAGTTCGCCCCGGAGAAACCCGGCCCCCAGCCTTCAGCCGAAACCAC
ACGCCACTTCCTCATGTCTGACCGGT,

which encodes the amino acid sequence |NheI|-CBRnt (1-413)-Ser-Gly-Leu-Lys-Ser-Arg-Arg-Ala-Leu-Asp-Ser-Ala-Barr2 (2-196)-|AgeI|:

[SEQ ID NO: 24]
|NheI|-
MVKREKNVIYGPEPLHPLEDLTAGEMLFRALRKHSHLPQALVDVVGDESL
SYKEFFEATVLLAQSLHNCGYKMNDVVSICAENNTRFFIPVIAAWYIGMI
VAPVNESYIPDELCKVMGISKPQIVFTTKNILNKVLEVQSRTNFIKRIII
LDTVENIHGCESLPNFISRYSDGNIANFKPLHFDPVEQVAAILCSSGTTG
LPKGVMQTHQNICVRLIHALDPRYGTQLIPGVTVLVYLPFFHAFGFHITL
GYFMVGLRVIMFRRFDQEAFLKAIQDYEVRSVINVPSVILFLSKSPLVDK
YDLSSLRELCCGAAPLAKEVAEVAAKRLNLPGIRCGFGLTESTSAIIQTL
GDEFKSGSLGRVTPLMAAKIADRETGKALGPNQVGELCIKGPMVSKGYVN
NVEATKEAIDDDG-SGLKSRRALDSA-
GEKPGTRVFKKSSPNCKLTVYLGKRDFVDHLDKVDPVDGVVLVDPDYLKD
RKVFVTLTCAFRYGREDLDVLGLSFRKDLFIATYQAFPPVPNPPRPPTRL
QDRLLRKLGQHAHPFFFFTIPQNLPCSVTLQPGPEDTGKACGVDFEIRAF
CAKSLEEKSHKRNSVRLVIRKVQFAPEKPGPQPSAETTREEFLMSDR-
|AgeI|,

where |NheI| and |AgeI| refer to the extra nucleotides outside the coding region for the amino acid sequence listed that encode the restriction enzyme digestion sites.

The ligation reactions resulted in the generation of DNA encoding the following protein sequences:

DOR-CBGct ligation product: Signal peptide +
FLAG tag-DOR-Gly-Ser-CBGct (395-542)-STOP, which
has the amino acid sequence
[SEQ ID NO: 19]
MKTIIALSYLFCLVFADYKDDDDA-
MELVPSARAELQSSPLVNLSDAFPSAFPSAGANASGSPGARSASSLALAI
AITALYSAVCAVGLLGNVLVMFGIVRYTKLKTATNIYIFNLALADALATS
TLPFQSAKYLMETWPFGELLCKAVLSIDYYNMFTSIFTLTMMSVDRYIAV
CIAPVKALDFRTPAKAKLI
NICIWVLASGVGVPIMVMAVTQPRDGAVVCMLQFPSPSWYWDTVTKICVF
LFAFVVPILIITVCYGLMLLRLRSVRLLSGSKEKDRSLRRITRMVLVVVG
AFVVCWAPIHIFVIVWTLVDINRRDPLVVAALEILCIALGYANSSLNPVL
YAFLDENFKRCFRQLCRTPCGRQEPGSLRRPRQATTRERVTACTPSDGPG
GGAAA-GS-SKGYVNNVEATKEAIDDDGWLHSGDFGYYDEDEEIFYVVDR
YKELIKYKGSQVAPAELEEILLKNPCIRDVAVVGIPDLEAGELPSAFVVK
QPGKEITAKEVYDYLAERVSHTKYLRGGVRFVDSIPRNVTGKITRKELLK
QLLEKAGG-STOP

CBGnt-Barr1 ligation product: CBGnt (1-413)-Ser-Gly-Leu-Lys-Ser-Arg-Arg-Ala-Leu-Asp-Ser-Ala-Barr1, which has the amino acid sequence:

[SEQ ID NO: 20]
MVKREKNVIYGPEPLHPLEDLTAGEMLFRALRKHSHLPQALVDVVGDESL
SYKEFFEATVLLAQSLHNCGYKMNDVVSICAENNTRFFIPVIAAWYIGMI
VAPVNESYIPDELCKVMGISKPQIVFTTKNILNKVLEVQSRTNFIKRIII
LDTVENIHGCESLPNFISRYSDGNIANFKPLHFDPVEQVAAILCSSGTTG
LPKGVMQTHQNICVRLIHALDPRVGTQLIPGVTVLVYLPFFHAFGFSITL
GYFMVGLRVIMFRRFDQEAFLKAIQDYEVRSVINVPSVILFLSKSPLVDK
YDLSSLRELCCGAAPLAKEVAEVAAKRLNLPGIRCGFGLTESTSANIHSL
GDEFKSGSLGRVTPLMAAKIADRETGKALGPNQVGELCIKGPMVSKGYVN
NVEATKEAIDDDG-SGLKSRRALDSA-
GDKGTRVFKKASPNGKLTVYLGKRDFVDHIDLVDPVDGVVLVDPEYLKER
RVYVTLTCAFRYGREDLDVLGLTFRKDLFVANVQSFPPAPEDKKPLTRLQ
ERLIKKLGEHAYPFTFEIPPNLPCSVTLQPGPEDTGKACGVDYEVKAFCA
ENLEEKIHKRNSVRLVIRKVQYAPERPGPQPTAETTRQFLMSDKPLHLEA
SLDKEIYYHGEPISVNVHVTNNTNKTVKKIKISVRQYADICLFNTAQYKC
PVAMEEADDTVAPSSTFCKVYTLTPFLANNREKRGLALDGKLKHEDTNLA
SSTLLREGANREILGIIVSYKVKVKLVVSRGGDVAVELPFTLMHPKPKEE
PPHREVPENETPVDTNLIELDTNDDDIVFEDFARQRLKGMKDDKEEEEDG
TGSPQLNNR-STOP

CBRnt-Barr2 ligation product: CBRnt (1-413)-Ser-Gly-Leu-Lys-Ser-Arg-Arg-Ala-Leu-Asp-Ser-Ala-Barr2, which has the amino acid sequence:

[SEQ ID NO: 21]
MVKREKNVIYGPEPLHPLEDLTAGEMLFRALRKHSHLPQALVDVVGDESL
SYKEFFEATVLLAQSLHNCGYKMNDVVSICAENNTRFFIPVIAAWYIGMI
VAPVNESYIPDELCKVMGISKPQIVFTTKNILNKVLEVQSRTNFIKRIII
LDTVENIHGCESLPNFISRYSDGNIANFKPLHFDPVEQVAAILCSSGTTG
LPKGVMQTHQNICVRLIHALDPRYGTQLIPGVTVLVYLPFFHAFGFHITL
GYFMVGLRVIMFRRFDQEAFLKAIQDYEVRSVINVPSVILFLSKSPLVDK
YDLSSLRELCCGAAPLAKEVAEVAAKRLNLPGIRCGFGLTESTSAIIQTL
GDEFKSGSLGRVTPLMAAKIADRETGKALGPNQVGELCIKGPMVSKGYVN
NVEATKEAIDDDG-SGLKSRRALDSA-
GEKPGTRVFKKSSPNCKLTVYLGKRDFVDHLDKVDPVDGVVLVDPDYLKD
RKVFVTLTCAFRYGREDLDVLGLSFRKDLFIATYQAFPPVPNPPRPPTRL
QDRLLRKLGQHAHPFFFTIPQNLPCSVTLQPGPEDTGKACGVDFEIRAFC
AKSLEEKSHKRNSVRLVIRKVQFAPEKPGPQPSAETTRHFLMSDRSLHLE
ASLDKELYYHGEPLNVNVHVTNNSTKTVKKIKVSVRQYADICLFSTAQYK
CPVAQLEQDDQVSPSSTFCKVYTITPLLSDNREKRGLALDGKLKEEEDTN
LASSTIVKEGANKEVLGILVSYRVKVKLVVSRGGDVSVELPFVLMHPKPH
DHIPLPRPQSAAPETDVPVDTNLIEFDTNYATDDDIVFEDFARLRLKGMK
DDDYDDQLC-STOP

### Example 2

### Running a ClickArr Assay with Cloning Products from Example 1

EK293 cells were seeded in an appropriate plate in full growth medium (Eagle's minimum essential medium (EMEM)+10% fetal bovine serum (FBS)). Typically, 5×10⁵ cells were seeded per well in a standard six-well plate. The next day, cells were transfected using X-tremeGENE™ 9 DNA Transfection Reagent (Roche Diagnostics GmbH, Mannheim, Germany) at a 1:1:1 mass ratio of DOR-CBGct:CBGnt-Barr1:CBRnt-Barr2 DNA. Salmon sperm DNA (Invitrogen, Carlsbad, Calif.) was used as a substitute for an excluded construct when HEK293 cells were doubly transfected with DOR and a single arrestin construct. Transfections were optimized and carried out according to the manufacturer's guidelines.

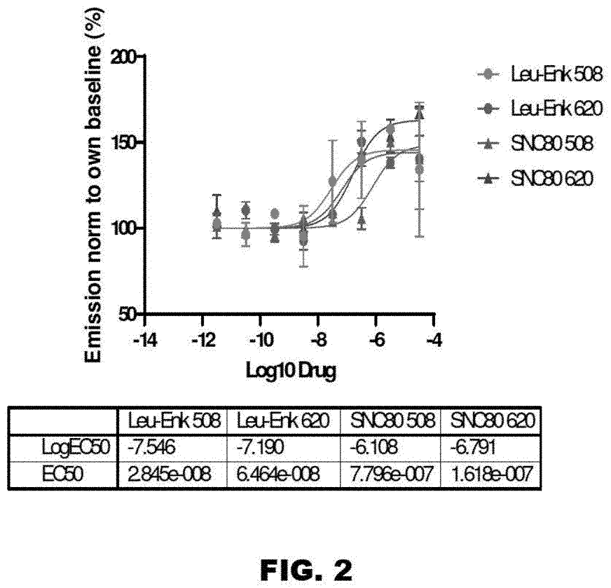

Around 38-40 hours post-transfection, cells were rinsed with DPBS and digested with trypsin-EDTA solution (Gibco). Cells were dissociated in 2× volume Opti-MEM (Gibco), pelleted at 300×g for 5.5 min and resuspended to 2-3×10⁶/mL in Opti-MEM. Cells (7.5 μL suspension/well) were seeded on a 384-well plate, spun, covered with AeraSeal (Millipore-Sigma) to prevent drying out, and put in an incubator at 37° C./5% CO₂ for 30 min. Plates were spun briefly to bring down any condensation on the upper part of the plate. D-Luciferin (7.5 μL of 2 mM solution in assay buffer (HBSS (Gibco) with 20 mM HEPES)) was added to each well. Plates were centrifuged briefly, re-covered, and placed back in the incubator at 37° C./5% CO₂ for 30 min. Drug solutions were prepared in assay buffer at 4× the desired final concentration. Plates were removed from the incubator and centrifuged briefly. Drug solution (5 μL) was added to each well. Plates were spun, re-covered, and placed back in the incubator at 37° C./5% CO₂ for 30 min. A plate reader (Biotek Synergy4 plate reader with 508/20 and 620/10 EM filters) was pre-heated to 37° C. and luminescence (0.5 sec integration time) was measured. The plate reader was controlled using Gen5 v2.04 software. The data were analyzed and plotted using GraphPad Prism 9 software. Dose-response curves were analyzed using the three parameter “log(agonist) vs. response” algorithm in Prism 9. An example of a dose-response curve obtained in accordance with the method is shown in FIG. 2. FIG. 2 is an exemplary dose-response curve obtained with two δ opioid receptor (DOR) agonists (Leu-Enk (leucine-enkephalin; YGGFL [SEQ ID NO: 15]) and SNC80 [(+)-4-[(αR)-α-((2S,5R)-4-Allyl-2,5-dimethyl-1-piperazinyl)-3-methoxybenzyl]-N,N-diethylbenzamide]).

### Example 3

### Optimizing Constructs

To optimize construct performance, a library of constructs having the linker sequences from Misawa et al. was screened (Anal Chem 82: 2552-2560 (2010)). To mutate the original constructs and generate the linker library, the NEBuilder HiFi DNA Assembly kit (New England Biolabs) was used. Primers encoding the desired linkers and flanking regions, which overlap sequences of DOR, Barr1, Barr2, CBG, and CBR, were designed according to the manufacturer's guidelines. Two sets of primers were ordered for each construct to test different termini of the luciferase fragments. For DOR-CBGct, primers were ordered to include the fragments CBG (395-542) and CBG (394-542). For CBGnt-Barr1, CBG (1-413) and CBG (1-415) were tested. For CBRnt-Barr2, CBR (1-413) and CBR (1-415) were tested. The linker sequences tested are as follows (in single-letter amino acid abbreviations):

DOR-linker-CBGct:
GS (original linker)
[SEQ ID NO: 1]
GSSGGGG
[SEQ ID NO: 2]
GSSGGGGSGGGG
[SEQ ID NO: 3]
GSSGGGGSGGGGSGGGG
[SEQ ID NO: 4]
GSSGGGGSGGGGSGGGGSGGGG
[SEQ ID NO: 5]
GSSGGGGSGGGGSGGGGSGGGGSGGGG
CBGnt-linker-Barr1 and CBRnt-linker-Barr2:
(original linker; [SEQ ID NO: 6])
SGLKSRRALDSA
[SEQ ID NO: 7]
GGGGGS
[SEQ ID NO: 8]
SGGGGSGGGGS
[SEQ ID NO: 9]
SGGGGSGGGGSGGGGS
[SEQ ID NO: 10]
SGGGGSGGGGSGGGGSGGGGS
[SEQ ID NO: 11]
SGGGGSGGGGSGGGGSGGGGSGGGGS

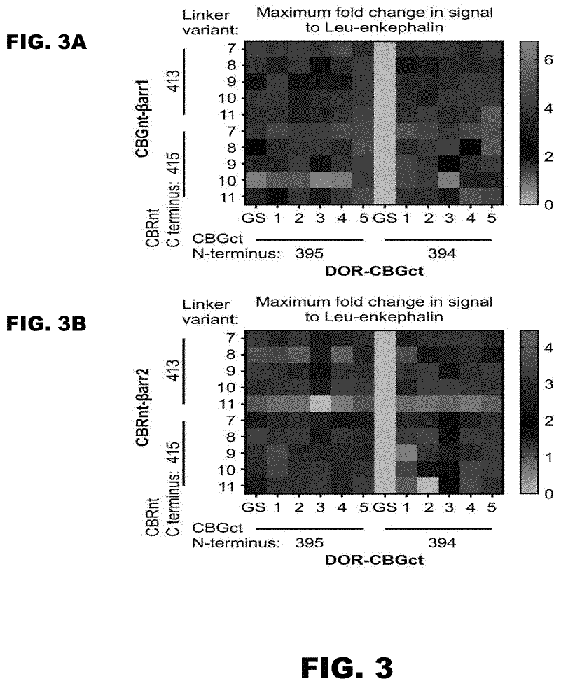

ClickArr assays were ran as described in Example 2 above. As shown in FIGS. 3A and 3B, the ClickArr assay components provide more than sufficient optimization of constructs for the DOR. FIG. 3A is a heat map showing the fold change in signal in response to Leu-enkephalin, the endogenous agonist for DOR, for cells transfected with the indicated DOR-CBGct construct and CBGnt-Barr1. FIG. 3B is a heat map showing the fold change in signal in response to Leu-enkephalin, the endogenous agonist for DOR, for cells transfected with the indicated DOR-CBGct construct and CBRnt-Barr2.

Optimized constructs include:

Signal peptide + FLAG tag-DOR-
GSSGGGGSGGGGSGGGGSGGGG-CBGct(395-542):
[SEQ ID NO: 25]
MKTIIALSYIFCLVFADYKDDDDA-
MELVPSARAELQSSPLVNLSDAFPSAFPSAGANASGSPGARSASSLAL
AIAITALYSAVCAVGLLGNVLVMFGIVRYTKLKTATNIYIFNLALADA
LATSTLPFQSAKYLMETWPFGELLCKAVLSIDYYNMFTSIFTLTMMSV
DRYIAVCIAPVKALDFRTPAKAKLINICIWVLASGVGVPIMVMAVTQP
RDGAVVCMLQFPSPSWYWDTVTKICVFLFAFVVPILIITVCYGLMLLR
LRSVRLLSGSKEKDRSLRRITRMVLVVVGAFVVCWAPIHIFVIVWTLV
DINRRDPLVVAALHLCIALGYANSSLNPVLYAFLDENFKRCFRQLCRT
PCGRQEPGSLRRPRQATTRERVTACTPSDGPGGGAAA-
GSSGGGGSGGGGSGGGGSGGGG-
SKGYVNNVEATKEAIDDDGWLHSGDFGYYDEDEHFYVVDRYKELIKYK
GSQVAPAELEEILLKNPCIRDVAVVGIPDLEAGELPSAFVVKQPGKEI
TAKEVYDYLAERVSHTKYLRGGVRFVDSIPRNVTGKITRKELLKQLLE
KAGG-STOP,
CBGct(1-415)-SGGGGSGGGGSGGGGSGGGGS-Barr1:
[SEQ ID NO: 26]
MVKREKNVIYGPEPLEEPLEDLTAGEMLFRALRKHSHLPQALVDVVGD
ESLSYKEFFEATVLLAQSLHNCGYKMNDVVSICAENNTRFFIPVIAAW
YIGMIVAPVNESYIPDELCKVMGISKPQIVFTTKNILNKVLEVQSRTN
FIKRIIILDTVENIFIGCESLPNFISRYSDGNIANFKPLHFDPVEQVA
AILCSSGTTGLPKGVMQTHQNICVRLIHALDPRVGTQLIPGVTVLVYL
PFFHAFGFSITLGYFMVGLPKGVIMFRRFDQEAFLKAIQDYEVRSVIN
VPSVILFLSKSPLVDKYDLSSLRELCCGAAPLAKEVAEVAAKRLNLPG
IRCGFGLTESTSANIHSLGDEFKSGSLGRVTPLMAAKIADRETGKALG
PNQVGELCIKGPMVSKGYVNNVEATKEAIDDDGWL-SGGGGSGGGGSG
GGGSGGGGS-GDKGTRVFKKASPNGKLTVYLGKRDFVDHIDLVDPVDG
VVLVDPEYLKERRVYVTLTCAFRYGREDLDVLGLTFRKDLFVANVQSF
PPAPEDKKPLTRLQERLIKKLGEHAYPFTFEIPPNLPCSVTLQPGPED
TGKACGVDYEVKAFCAENLEEKIHKRNSVRLVIRKVQYAPERPGPQPT
AETTRQFLMSDKPLIALEASLDKEIYYHGEPISVNVHVTNNTNKTVKK
IKISVRQYADICLFNTAQYKCPVAMEEADDTVAPSSTFCKVYTLTPFL
ANNREKRGLALDGKLKEEEDTNLASSTLLREGANREILGIIVSYKVKV
KLVVSRGGDVAVELPFTLMHPKPKEEPPHREVPENETPVDTNLIELDT
NDDDIVFEDFARQRLKGMKDDKEEEEDGTGSPQLNNR-STOP,
and
CBRnt(1-413)-SGGGGSGGGGS-Barr2:
[SEQ ID NO: 27]
MVKREKNVIYGPEPLEEPLEDLTAGEMLFRALRKHSIALPQALVDVVG
DESLSYKEFFEATVLLAQSLHNCGYKMNDVVSICAENNTRFFIPVIAA
WYIGMIVAPVNESYIPDELCKVMGISKPQIVFTTKNILNKVLEVQSRT
NFIKRIIILDTVENIHGCESLPNFISRYSDGNIANFKPLHFDPVEQVA
AILCSSGTTGLPKGVMQTHQNICVRLIHALDPRYGTQLIPGVTVLVYL
PFFHAFGFHITLGYFMVGLRVIMFRRFDQEAFLKAIQDYEVRSVINVP
SVILFLSKSPLVDKYDLSSLRELCCGAAPLAKEVAEVAAKRLNLPGIR
CGFGLTESTSAIIQTLGDEFKSGSLGRVTPLMAAKIADRETGKALGPN
QVGELCIKGPMVSKGYVNNVEATKEAIDDDG-SGGGGSGGGGS-GEKP
GTRVFKKSSPNCKLTVYLGKRDFVDIALDKVDPVDGVVLVDPDYLKDR
KVFVTLTCAFRYGREDLDVLGLSFRKDLFIATYQAFPPVPNPPRPPTR
LQDRLLRKLGQHAHPFFFTIPQNLPCSVTLQPGPEDTGKACGVDFEIR
AFCAKSLEEKSHKRNSVRLVIRKVQFAPEKPGPQPSAETTREEFLMSD
RSLHLEASLDKELYYHGEPLNVNVHVTNNSTKTVKKIKVSVRQYADIC
LFSTAQYKCPVAQLEQDDQVSPSSTFCKVYTITPLLSDNREKRGLALD
GKLKHEDTNLASSTIVKEGANKEVLGILVSYRVKVKLVVSRGGDVSVE
LPFVLMHPKPHDHIPLPRPQSAAPETDVPVDTNLIEFDTNYATDDDIV
FEDFARLRLKGMKDDDYDDQLC-STOP.

### Example 4

### General Application and Suitability of ClickArr Method to Drug Screening, Different DOR Agonists, Non-Kinetic vs. Kinetic Experiments, and Different GPCRs

D-luciferin sodium salt was purchased from GoldBio. Leu-Enk (Leu-enkephalin, YGGFL [SEQ ID NO: 15]), SNC80 (4-[(R)-[(2S,5R)-2,5-dimethyl-4-prop-2-enylpiperazin-1-yl]-(3-methoxyphenyl)methyl]-N,N-diethylbenzamide), TAN67 (SB 205607, (R*,S*)-(±)-2-Methyl-4aa-(3-hydroxyphenyl)-1,2,3,4,4a,5,12,12aa-octahydroquinolino[2,3,3-g]isoquinoline dihydrobromide), ARM390 (AR-M 1000390, N,N-Diethyl-4-(phenyl-4-piperidinylidenemethyl)-benzamide hydrochloride), Dynorphin17 (YGGFLRRIRPKLKWDNQ [SEQ ID NO: 16]), and Angiotensin II (DRVYIHPF [SEQ ID NO: 17]) were purchased from Tocris Biosciences. ADL5859 (N,N-diethyl-4-(5-hydroxyspiro[chromene-2,4′-piperidine]-4-yl)benzamide hydrochloride) and UNC9994 (5-(3-(4-(2,3-dichlorophenyl)piperidin-1-yl)propoxy)benzo[d]thiazole hydrochloride) were purchased from AxonMedChem.

Constructs utilizing the DOR, Barr1, and Barr2 were cloned according to Examples 1 and 2 above. Additionally, to test whether the method was extendable to other GPCRs, the DNA encoding the optimal linker+CBGct fragment for the DOR (GSSGGGGSGGGGSGGGGSGGGG—CBGct (res. 395-542)-STOP), which has the amino acid sequence:

[SEQ ID NO: 18]
GSSGGGGSGGGGSGGGGSGGGG-
SKGYVNNVEATKEAIDDDGWLHSGDFGYYDEDEHFYVVDRYKELIKYKG
SQVAPAELEEILLKNPORDVAVVGIPDLEAGELPSAFVVKQPGKEITAK
EVYDYLAERVSHTKYLRGGVRFVDSIPRNVTGKITRKELLKQLLEKAG
G-STOP,

was amplified by PCR and fused to PCR-amplified DNA fragments encoding either the kappa opioid receptor (KOR), dopamine D2 receptor (D2R), or the angiotensin type 1 receptor (AGTR1) using the HiFi cloning kit into the original DOR-linker-CBGct backbone digested with NheI/XbaI (New England Biolabs). The mouse KOR sequence was used and includes an influenza signal peptide for expression and a FLAG tag for future labeling studies. For D2R and AGTR1, the human sequences from cdna.org were used; the AGTR1 sequence retains the 3xHA-tag for future labeling studies.

The construct sequences are thus:

Signal peptide + FLAG tag-KOR-
GSSGGGGSGGGGSGGGGSGGGG-CBGct(395-542):
[SEQ ID NO: 28]
MKTIIALSYIFCLVFADYKDDDDA-
MESPIQIFRGDPGPTCSPSACLLPNSSSWFPNWAESDSNGSVGSEDQQL
ESAHISPAIPVIITAVYSVVFVVGLVGNSLVMFVIIRYTKMKTATNIYI
FNLALADALVTTTMPFQSAVYLMNSWPFGDVLCKIVISIDYYNMFTSIF
TLTMMSVDRYIAVCHPVKALDFRTPLKAKIINICIWLLASSVGISAIVL
GGTKVREDVDVIECSLQFPDDEYSWWDLFMKICVFVFAFVIPVLIIIVC
YTLMILRLKSVRLLSGSREKDRNLRRITKLVLVVVAVFIICWTPIHIFI
LVEALGSTSHSTAALSSYYFCIALGYTNSSLNPVLYAFLDENFKRCFRD
FCFPIKMRMERQSTNRVRNTVQDPASMRDVGGMNKPV-GSSGGGGSGGG
GSGGGGSGGGG-SKGYVNNVEATKEMDDDGWLHSGDFGYYDEDEHFYVV
DRYKELIKYKGSQVAPAELEELLIKNPCIRDVAVVGIPDLEAGELPSAF
VVKQPGKEITAKEVYDYLAERVSHTKYLRGGVRFVDSIPRNVTGKITRK
ELLKQLLEKAGG-STOP
D2R-GSSGGGGSGGGGSGGGGSGGGG-CBGct(395-542):
[SEQ ID NO: 29]
MDPLNLSWYDDDLERQNWSRPFNGSDGKADRPHYNYYATLLTLLIAVIV
FGNVLVCMAVSREKALQTTTNYLIVSLAVADLLVATLVMPWVVYLEVVG
EWKFSRIHCDIFVTLDVMMCTASILNLCAISIDRYTAVAMPMLYNTRYS
SKRRVTVMISIVWVLSFTISCPLLFGLNNADQNECIIANPAFVVYSSIV
SFYVPFIVTLLVYIKIYIVLRRRRKRVNTKRSSRAFRAHLRAPLKGNCT
HPEDMKLCTVIMKSNGSFPVNRRRVEAARRAQELEMEMLSSTSPPERTR
YSPIPPSHHQLTLPDPSHHGLHSTPDSPAKPEKNGHAKDRPKIAKIFEI
QTMPNGKTRTSLKTMSRRKLSQQKEKKATQMLAIVLGVFIICWLPFFIT
HILNIHCDCNIPPVLYSAFTWLGYVNSAVNPIIYTTFNIEFRKAFLKIL
HC-GSSGGGGSGGGGSGGGGSGGGG-AKGYVNNVEATKEMDDDGWLHSG
DFGYYDEDEHFYVVDRYKELIKYKGSQVAPAELEELLKNPCIRDVAVVG
IPDLEAGELPSAFVVKQPGKEITAKEVYDYLAERVSHTKYLRGGVRFVD
SIPRNVTGKITRKELLKQLLEKAGG-STOP
3xHA-AGTR1(2-360)-GSSGGGGSGGGGSGGGGSGGGG-
CBGct(395-542):
[SEQ ID NO: 30]
MYPYDVPDYAYPYDVPDYAYPYDVPDYA-
DILNSSTEDGIKRIQDDCPKAGRHNYIFVMIPTLYSIIFVVGIFGNSLV
VIVIYFYMKLKTVASVFLLNLALADLCFLLTLPLWAVYTAMEYRWPFGN
YLCKIASASVSFNLYASVFLLTCLSIDRYLAIVHPMKSRLRRTMLVAKV
TCIIIWLLAGLASLPAIIHRNVFFIENTNITVCAFHYESQNSTLPIGLG
LTKNILGFLFPFLIILTSYTLIWKALKKAYEIQKNKPRNDDIFKIIMAI
VLFFFFSWIPHQIFTFLDVLIQLGIIRDCRIADIVDTAMPITICIAYFN
NCLNPLFYGFLGKKFKRYFLQLLKYIPPKAKSHSNLSTKMSTLSYRPSD
NVSSSTKKPAPCFEVE-GSSGGGGSGGGGSGGGGSGGGG-SKGYVNNVE
ATKEAIDDDGWLHSGDFGYYDEDEHFYVVDRYKELIKYKGSQVAPAELE
EILLKNPCIRDVAVVGIPDLEAGELPSAFVVKQPGKEITAKEVYDYLAE
RVSHTKYLRGGVRFVDSIPRNVTGKITRKELLKQLLEKAGG-STOP

On day 1, HEK293 cells were seeded in an appropriate plate in full growth medium (EMEM+10% FBS). Typically, 5×10⁵ cells were seeded per well in a standard 6-well plate. On day 2, cells were transfected with the appropriate transfection method. XtremeGene9 (Roche) was used with a 1:1:1 or a 2:1:1 mass ratio of DOR-CBGct:CBGnt-Barr1:CBRnt-Barr2 DNA. For double transfection of DOR with a single arrestin construct, salmon sperm DNA (Invitrogen) was substituted for the construct being excluded. Transfections were optimized and carried out according to the manufacturer's guidelines.

On day 4, the following assay procedure was started 38-40 hours following transfection:
1. Rinse cells with DPBS and digest cells with trypsin-EDTA solution (Gibco).
2. Dissociate cells in 2× volume Opti-MEM (Gibco).
3. Pellet cells at 5.5 minutes/300×g and resuspend to 2-3×10⁶/mL in Opti-MEM.
4. Seed 7.5 μL cell suspension/well of a 384-well plate.
5. Spin, cover with AeraSeal (Millipore-Sigma) to prevent drying out, and put in 37 C incubator for 30 minutes.
6. Make up a 2 mM luciferin solution using either D-Luciferin frozen stocks or powder in Assay buffer (AB).
   a. Aliquots of a 100 mM Na-luciferin stock made in assay buffer (AB, 980 μL HBSS (Gibco), 20 μL 1M HEPES (Gibco)) can be frozen at −80° C. and used for at least four weeks.
7. Take out plate and briefly spin to bring down any moisture condensing on the upper part of the plate.
8. Pipette 7.5 μL of 2 mM D-luciferin mix in each well and briefly centrifuge. Recover plate and put back in 37 C and incubate for 30 minutes.
   a. Prepare drug solutions in AB at 4 times the desired final concentration.
      For assays with a single timepoint, such as dose-response curves:
9. Take out plate and briefly centrifuge. Add 5 uL of drug solution to each well. Spin plate, reseal, and incubate for 30 minutes at 37 C.
10. Pre-heat plate reader to 37 C.
11. After incubation is complete, read plate with 0.5 s integration time.
   For assays with kinetic reads (multiple time points) beginning at 30 minutes post-drug addition:
9. Take out plate and briefly centrifuge. Add 5 uL of drug solution to each well. Spin plate, reseal, and incubate for 30 minutes at 37° C.
10. Pre-heat plate reader to 37° C. Set plate reader to read at desired time intervals and 0.5 s integration time.
11. After incubation is complete, read plate.
   For assays with kinetic reads (multiple time points) beginning at 0 minutes post-drug addition:
9. Pre-heat plate reader to 37° C. and measure luminescence (0.5 s integration time). Set plate reader to read at indicated time intervals.
   9a. For this type of kinetic read, it is recommended to perform a five-minute kinetic baseline read in the plate reader to allow the plate to equilibrate to the plate reader's temperature.
   9b. Program the plate reader to pause the kinetic read following acquisition of the baseline, allowing for drug addition. Alternatively, if the plate reader being used has a suitable integrated pipettor system, follow the manufacturer's guidelines on implementing that tool instead of manual drug addition. Drug preparation needs to be performed in a way that is suitable for the plate reader's pipettor system, if being used. In this study, pipetting was performed manually.
10. Take out plate and briefly centrifuge. Place in plate reader and acquire baseline.
11. Set aliquots of drug dilutions to warm in a 37° C. water bath so as not to change the temperature of the cell suspension upon addition.
   11a. If a large aliquot is needed, it may be necessary to place the drug solution in the water bath at an earlier step to ensure equilibration.
12. Once acquisition pauses following collection of the baseline data, work quickly to add 5 μL of drug solution to each well. Spin plate and return to plate reader for the remainder of the kinetic acquisition.
12a. The inclusion of wells that receive only vehicle (assay buffer plus drug solvent) is recommended to normalize the data against fluctuations in brightness due to temperature and consumption of D-luciferin.

A Biotek Synergy4 plate reader with 508/20 and 620/10 EM filters was used in all experiments. The plate reader was controlled using Gen2 software. The data were analyzed and plotted using GraphPad Prism 9 software. Dose-response curves were analyzed using the three parameter “log(agonist) vs. response” algorithm in Prism 9.

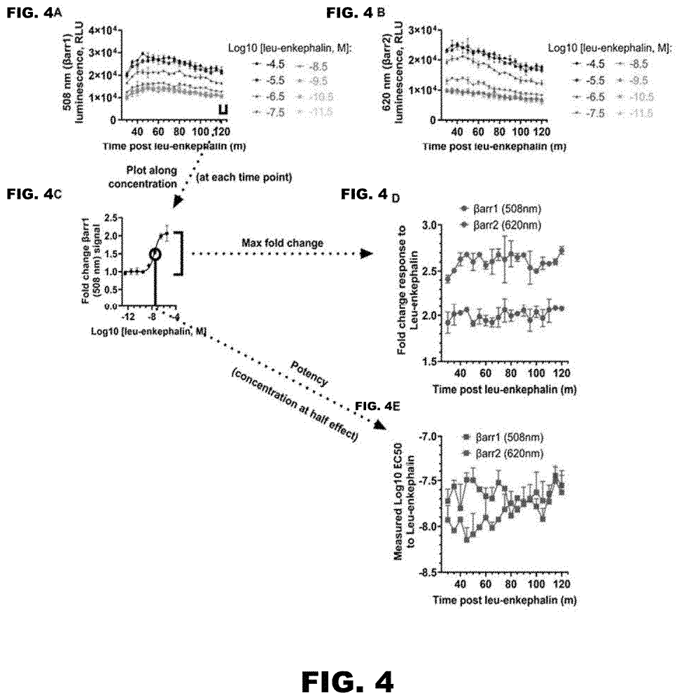

Cells triply transfected with optimized DOR, Barr1, and Barr2 constructs were treated with a 10⁷× range of concentrations of leu-enkephlin and emissions from Barr1 and Barr2 were collected over time. The results are shown in FIGS. 4A and 4B, respectively. FIG. 4A is a graph of time post-leu-enkephalin (m) vs. 508 nm (Barr1) luminescence (RLU). FIG. 4B is a graph of time post-leu-enkephalin (m) vs. 620 nm (Barr2) luminescence (RLU). Data from FIGS. 4A and 4B were converted to yield common parameters for evaluating new drugs as shown in FIG. 4C, which is a graph of log10 [leu-enkephalin (M)] vs. fold change Barr 1 (508 nm) signal. Each time point can be re-plotted as effect vs. concentration, and this dose-response curve can be fit to yield the efficacy (fold change) and the potency (concentration at half effect). The analysis in FIG. 4C carried out at each timepoint in FIGS. 4A and 4B can be used to yield graphs of the efficacy and potency over time as shown in FIGS. 4D and 4E, respectively. FIG. 4D is a graph of time post-leu-enkephalin (m) vs. fold change response to leu-enkephalin. FIG. 4E is a graph of time post-leu-enkephalin (m) vs. measured log10 EC50 to leu-enkephalin. The results show that the ClickArr assay is stable in the indicated time range and can serve as a reliable drug-screening platform for searching for new therapeutics with β-arrestin isoform bias.

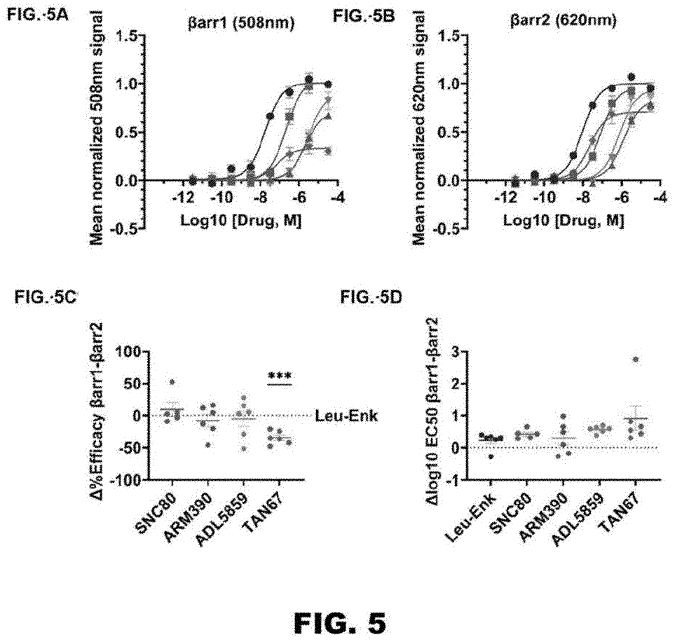

Cells were triply infected with optimized DOR, Barr1, and Barr2 constructs [SEQ ID NO: 25-27]. Various DOR agonists were assayed and their dose-response curves are shown in FIGS. 5A and 5B. FIG. 5A is a graph of log10 [drug (M)] vs. mean normalized 508 nm signal (Barr1). FIG. 5B is a graph of log10 [drug (M)] vs. mean normalized 620 nm signal (Barr2). Data were normalized to the standard leu-enkephalin (Leu-Enk), the endogenous agonist for the DOR. Curves in FIGS. 5A and 5B were fit and the difference in efficacy for each drug between Barr1 and Barr2 is shown in FIG. 5C, which is a graph of drug vs. Δ% efficacy Barr1-Barr2. When compared to Leu-Enk, TAN67 had a significant difference in efficacy (Δ% efficacy is the difference in maximum change in signal between low and high concentration of drug between each arrestin isoform, as a percentage of Leu-Enk). The data show that the ClickArr method can detect isoform bias at a GPCR. As shown in FIG. 5D, which is a graph of drug or standard (Leu-Enk) vs. Δlog10 EC50 Barr1-Barr2, the relative potency values (the concentration at half effect in the curves in FIGS. 5A and 5B are not significant between the standard (Leu-Enk) and the drugs tested, indicating that the bias in TAN67 is due to efficacy only. n=5-6 biological replicates.

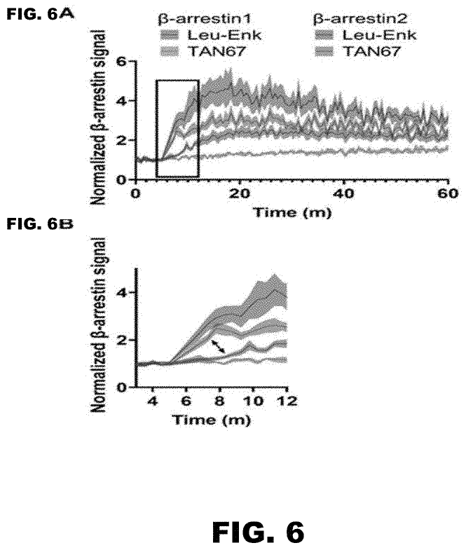

Kinetic traces of signals from cells treated with a saturating concentration (32 μM) of either leu-enkephalin or TAN67 are shown in FIGS. 6A and 6B. FIG. 6A is a graph of time (m) vs. normalized β-arrestin signal. FIG. 6B is a graph of time (m) vs. normalized β-arrestin signal, which is an enlargement of the portion of the graph outlined in FIG. 6A. n=6 replicates. The shaded area is mean±SEM. Recruitment of Barr1 is much slower than Barr2 regardless of drug, even though the signal from Barr1 recruited by leu-enkephalin eventually reaches similar levels as Barr2 recruited by TAN67. Together with data from FIG. 4, these data demonstrate the utility of the assay in reporting real-time kinetic differences in arrestin isoform recruitment to a GPCR across different agonists in living cells.

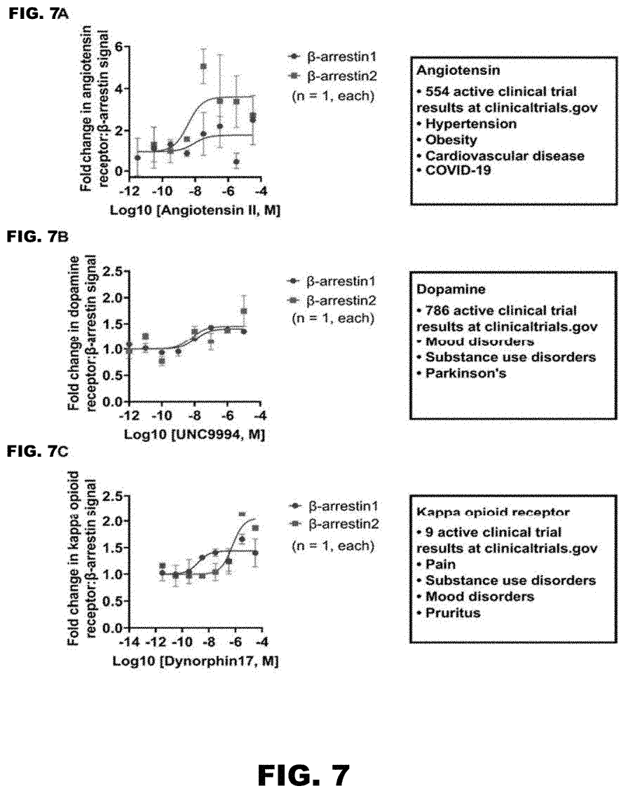

The click beetle green C-terminal fragment (CBGct) was fused to another neurobiological GPCR, the angiotensin receptor 1 (AGTR1) [SEQ ID NO: 30]. When expressed with the optimized arrestin constructs from the delta opioid receptor screen (FIG. 3), the construct showed a potential response to angiotensin II, an endogenous agonist for AGTR1. The results are shown in FIG. 7A, which is a graph of log10 [angiotensin II, M] vs. fold change in angiotensin receptor:β-arrestin signal. The CBGct fragment fused to the dopamine receptor 2 (D2R) [SEQ ID NO: 29] showed a response to UNC9994, which is a potent synthetic agonist for the D2R. The results are shown in FIG. 7B, which is a graph of log10 [UNC9994, M] vs. fold change in dopamine receptor: β-arrestin signal. The CBGct fragment fused to the kappa opioid receptor (KOR) [SEQ ID NO: 28] showed a response to dynorphin17, its endogenous agonist. The results are shown in FIG. 7C, which is a graph of log10 [dynorphin17, M] vs. fold change in kappa opioid receptor:β-arrestin signal. The results in FIGS. 7A-7C are noisy because they are single replicates from pilot experiments. Nonetheless, the β-arrestin curves appear to be as potent or more potent than published values for either angiotensin II at the AGTR1 (log10 (5.0 nM)˜−8.3; Wingler et al., Cell 176: 479-490 (2019)) or UNC9994 at the D2R (log10 (448 nM)˜−6.3; Allen et al., PNAS 108(45): 18488-18493 (2011)). The results are also close to the kappa opioid receptor (log10 (77 nM)˜−7.1; Dunn et al., ACS Chem Neurosci 10: 3590-3600 (2019)). These suggest with minimal optimization the applicability of the ClickArr assay toward reporting arrestin recruitment to a variety of GPCRs.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

The invention illustratively described herein may be suitably practiced in the absence of any element(s) or limitation(s), which is/are not specifically disclosed herein. Thus, for example, each instance herein of any of the terms “comprising,” “consisting essentially of,” and “consisting of” may be replaced with either of the other two terms. Likewise, the singular forms “a,” “an,” and “the” include plural references unless the context clearly dictates otherwise. Thus, for example, references to “the method” includes one or more methods and/or steps of the type, which are described herein and/or which will become apparent to those ordinarily skilled in the art upon reading the disclosure.

The terms and expressions, which have been employed, are used as terms of description and not of limitation. Where certain terms are defined and are otherwise described or discussed elsewhere in the “Detailed Description,” all such definitions, descriptions, and discussions are intended to be attributed to such terms. There also is no intention in the use of such terms and expressions of excluding any equivalents of the features shown and described or portions thereof. Furthermore, while subheadings may be used in the “Detailed Description,” such use is solely for ease of reference and is not intended to limit any disclosure made in one section to that section only; rather, any disclosure made under one subheading is intended to constitute a disclosure under each and every other subheading.

It is recognized that various modifications are possible within the scope of the claimed invention. Thus, although the present invention has been specifically disclosed in the context of preferred embodiments and optional features, those skilled in the art may resort to modifications and variations of the concepts disclosed herein. Such modifications and variations are considered within the scope of the invention as claimed herein.

### SEQUENCE LISTING

The written Sequence Listing below is the same as the above-described sequences and those provided in computer readable form encoded in a file, filed herewith, and herein incorporated by reference.

SEQ ID NO: 1 is GSSGGGG.
SEQ ID NO: 2 is GSSGGGGSGGGG.
SEQ ID NO: 3 is GSSGGGGSGGGGSGGGG.
SEQ ID NO: 4 is GSSGGGGSGGGGSGGGGSGGGG.
SEQ ID NO: 5 is GSSGGGGSGGGGSGGGGSGGGGSGGGG.
SEQ ID NO: 6 is SGLKSRRALDSA (original linker).
SEQ ID NO: 7 is GGGGGS.
SEQ ID NO: 8 is SGGGGSGGGGS.
SEQ ID NO: 9 is SGGGGSGGGGSGGGGS.
SEQ ID NO: 10 is SGGGGSGGGGSGGGGSGGGGS.
SEQ ID NO: 11 is SGGGGSGGGGSGGGGSGGGGSGGGGS.
SEQ ID NO: 12 is:
GCGGCCGCCAAGAACCCGGCAGTCTCCGTCGTCCCCGCCAGGCCACCACGCGTGAGCG
TGTCACTGCCTGCACCCCCTCCGACGGCCCGGGCGGTGGCGCCGCCGCCGGCAGTAGC
AAGGGTTATGTCAATAACGTTGAAGCTACCAAGGAGGCCATCGACGACGACGGCTGGT
TGCATTCTGGTGATTTTGGATATTACGACGAAGATGAGCATTTTTACGTCGTGGATCGT
TACAAGGAGCTGATCAAATACAAGGGTAGCCAGGTTGCTCCAGCTGAGTTGGAGGAGA
TTCTGTTGAAAAATCCATGCATTCGCGATGTCGCTGTGGTCGGCATTCCTGATCTGGAG
GCCGGCGAACTGCCTTCTGCTTTCGTTGTCAAGCAGCCTGGTAAAGAAATTACCGCCAA
AGAAGTGTATGATTACCTGGCTGAACGTGTGAGCCATACTAAGTACTTGCGTGGCGGC
GTGCGTTTTGTTGACTCCATCCCTCGTAACGTAACAGGCAAAATTACCCGCAAGGAGCT
GTTGAAACAATTGTTGGAGAAGGCCGGCGGTTGACCCTGGG, which encodes SEQ ID
NO: 22 (|NotI|-DOR (338-372)-Gly-Ser-CBGct (395-542)-STOP-
|PasI|(|NotI|-
GRQEPGSLRRPRQATTRERVTACTPSDGPGGGAAA-GS-
SKGYVNNVEATKEAIDDDGWLHSGDFGYYDEDEHFYVVDRYKELIKYKGSQVAPAELEEI
LLKNPCIRDVAVVGIPDLEAGELPSAFVVKQPGKEITAKEVYDYLAERVSHTKYLRGGVRF
VDSIPRNVTGKITRKELLKQLLEKAGG-STOP-|PasI|)).
SEQ ID NO: 13 is:
GCTAGCGCCGCCACCATGGTGAAGCGTGAGAAAAATGTCATCTATGGCCCTGAGCCTC
TCCATCCTTTGGAGGATTTGACTGCCGGCGAAATGCTGTTTCGTGCTCTCCGCAAGCAC
TCTCATTTGCCTCAAGCCTTGGTCGATGTGGTCGGCGATGAATCTTTGAGCTACAAGGA
GTTTTTTGAGGCAACCGTCTTGCTGGCTCAGTCCCTCCACAATTGTGGCTACAAGATGA
ACGACGTCGTTAGTATCTGTGCTGAAAACAATACCCGTTTCTTCATTCCAGTCATCGCC
GCATGGTATATCGGTATGATCGTGGCTCCAGTCAACGAGAGCTACATTCCCGACGAAC
TGTGTAAAGTCATGGGTATCTCTAAGCCACAGATTGTCTTCACCACTAAGAATATTCTG
AACAAAGTCCTGGAAGTCCAAAGCCGCACCAACTTTATTAAGCGTATCATCATCTTGG
ACACTGTGGAGAATATTCACGGTTGCGAATCTTTGCCTAATTTCATCTCTCGCTATTCA
GACGGCAACATCGCAAACTTTAAACCACTCCACTTCGACCCTGTGGAACAAGTTGCAG
CCATTCTGTGTAGCAGCGGTACTACTGGACTCCCAAAGGGAGTCATGCAGACCCATCA
AAACATTTGCGTGCGTCTGATCCATGCTCTCGATCCACGCGTGGGCACTCAGCTGATTC
CTGGTGTCACCGTCTTGGTCTACTTGCCTTTCTTCCATGCTTTCGGCTTTAGCATTACTTT
GGGTTACTTTATGGTCGGTCTCCGCGTGATTATGTTCCGCCGTTTTGATCAGGAGGCTTT
CTTGAAAGCCATCCAAGATTATGAAGTCCGCAGTGTCATCAACGTGCCTAGCGTGATCC
TGTTTTTGTCTAAGAGCCCACTCGTGGACAAGTACGACTTGTCTTCACTGCGTGAATTG
TGTTGCGGTGCCGCTCCACTGGCTAAGGAGGTCGCTGAAGTGGCCGCCAAACGCTTGA
ATCTTCCAGGGATTCGTTGTGGCTTCGGCCTCACCGAATCTACCAGCGCAAACATTCAC
TCTCTCGGGGATGAGTTTAAGAGCGGCTCTTTGGGCCGTGTCACTCCACTCATGGCTGC
TAAGATCGCTGATCGCGAAACTGGTAAGGCTTTGGGCCCGAACCAAGTGGGCGAGCTG
TGTATCAAAGGCCCTATGGTGAGCAAGGGTTATGTCAATAACGTTGAAGCTACCAAGG
AGGCCATCGACGACGACGGCAGCGGCCTGAAGAGCAGGAGGGCCCTGGACAGCGCTG
GCGACAAAGGGACCCGAGTGTTCAAGAAGGCCAGTCCAAATGGAAAGCTCACCGTCTA
CCTGGGAAAGCGGGACTTTGTGGACCACATCGACCTCGTGGACCCTGTGGATGGTGTG
GTCCTGGTCGATCCTGAGTATCTCAAAGAGCGGAGAGTCTATGTGACGCTGACCTGCG
CCTTCCGCTATGGCCGGGAGGACCTGGATGTCCTGGGCCTGACCTTTCGCAAGGACCTG
TTTGTGGCCAACGTACAGTCGTTCCCACCGGCCCCCGAGGACAAGAAGCCCCTGACGC
GGCTGCAGGAACGCCTCATCAAGAAGCTGGGCGAGCACGCTTACCCTTTCACCTTTGA
GATCCCTCCAAACCTTCCATGTTCTGTGACACTGCAGCCGGGGCCCGAAGACACGGGG
AAGGCTTGCGGTGTGGACTATGAAGTCAAAGCCTTCTGCGCGGAGAATTTGGAGGAGA
AGATCCACAAGCGCAATTCTGTGCGTCTGGTCATCCGGA, which encodes SEQ
ID NO: 23.
SEQ ID NO: 14 is:
GCTAGCGCCGCCACCATGGTAAAGCGTGAGAAAAATGTCATCTATGGCCCTGAGCCTC
TCCATCCTTTGGAGGATTTGACTGCCGGCGAAATGCTGTTTCGTGCTCTCCGCAAGCAC
TCTCATTTGCCTCAAGCCTTGGTCGATGTGGTCGGCGATGAATCTTTGAGCTACAAGGA
GTTTTTTGAGGCAACCGTCTTGCTGGCTCAGTCCCTCCACAATTGTGGCTACAAGATGA
ACGACGTCGTTAGTATCTGTGCTGAAAACAATACCCGTTTCTTCATTCCAGTCATCGCC
GCATGGTATATCGGTATGATCGTGGCTCCAGTCAACGAGAGCTACATTCCCGACGAAC
TGTGTAAAGTCATGGGTATCTCTAAGCCACAGATTGTCTTCACCACTAAGAATATTCTG
AACAAAGTCCTGGAAGTCCAAAGCCGCACCAACTTTATTAAGCGTATCATCATCTTGG
ACACTGTGGAGAATATTCACGGTTGCGAATCTTTGCCTAATTTCATCTCTCGCTATTCA
GACGGCAACATCGCAAACTTTAAACCACTCCACTTCGACCCTGTGGAACAAGTTGCAG
CCATTCTGTGTAGCAGCGGTACTACTGGACTCCCAAAGGGAGTCATGCAGACCCATCA
AAACATTTGCGTGCGTCTGATCCATGCTCTCGATCCACGCTACGGCACTCAGCTGATTC
CTGGTGTCACCGTCTTGGTCTACTTGCCTTTCTTCCATGCTTTCGGCTTTCATATTACTTT
GGGTTACTTTATGGTCGGTCTCCGCGTGATTATGTTCCGCCGTTTTGATCAGGAGGCTTT
CTTGAAAGCCATCCAAGATTATGAAGTCCGCAGTGTCATCAACGTGCCTAGCGTGATCC
TGTTTTTGTCTAAGAGCCCACTCGTGGACAAGTACGACTTGTCTTCACTGCGTGAATTG
TGTTGCGGTGCCGCTCCACTGGCTAAGGAGGTCGCTGAAGTGGCCGCCAAACGCTTGA
ATCTTCCAGGGATTCGTTGTGGCTTCGGCCTCACCGAATCTACCAGTGCGATTATCCAG
ACTCTCGGGGATGAGTTTAAGAGCGGCTCTTTGGGCCGTGTCACTCCACTCATGGCTGC
TAAGATCGCTGATCGCGAAACTGGTAAGGCTTTGGGCCCGAACCAAGTGGGCGAGCTG
TGTATCAAAGGCCCTATGGTGAGCAAGGGTTATGTCAATAACGTTGAAGCTACCAAGG
AGGCCATCGACGACGACGGCAGCGGCCTGAAGAGCAGGAGGGCCCTGGACAGCGCTG
GGGAGAAACCCGGGACCAGGGTCTTCAAGAAGTCGAGCCCTAACTGCAAGCTCACCGT
GTACTTGGGCAAGCGGGACTTCGTAGATCACCTGGACAAAGTGGACCCTGTAGATGGC
GTGGTGCTTGTGGACCCTGACTACCTGAAGGACCGCAAAGTGTTTGTGACCCTCACCTG
CGCCTTCCGCTATGGCCGTGAAGACCTGGATGTGCTGGGCTTGTCCTTCCGCAAAGACC
TGTTCATCGCCACCTACCAGGCCTTCCCCCCGGTGCCCAACCCACCCCGGCCCCCCACC
CGCCTGCAGGACCGGCTGCTGAGGAAGCTGGGCCAGCATGCCCACCCCTTCTTCTTCAC
CATACCCCAGAATCTTCCATGCTCCGTCACACTGCAGCCAGGCCCAGAGGATACAGGA
AAGGCCTGCGGCGTAGACTTTGAGATTCGAGCCTTCTGTGCTAAATCACTAGAAGAGA
AAAGCCACAAAAGGAACTCTGTGCGGCTGGTGATCCGAAAGGTGCAGTTCGCCCCGGA
GAAACCCGGCCCCCAGCCTTCAGCCGAAACCACACGCCACTTCCTCATGTCTGACCGGT
which encodes SEQ ID NO: 24.
SEQ ID NO: 15 is Tyr-Gly-Gly-Phe-Leu.
SEQ ID NO: 16 is Tyr-Gly-Gly-Phe-Leu-Arg-Arg-Ile-Arg-Pro-Lys-
Leu-Lys-Trp-Asp-Asn-Gln.
SEQ ID NO: 17 is Asp-Arg-Val-Tyr-Ile-His-Pro-Phe.
SEQ ID NO: 18 is GSSGGGGSGGGGSGGGGSGGGG - CBGct (res. 395-542)-
STOP
(GSSGGGGSGGGGSGGGGSGGGG-
SKGYVNNVEATKEAIDDDGWLHSGDFGYYDEDEHFYVVDRYKELIKYKGSQVAPAELEEI
LLKNPCIRDVAVVGIPDLEAGELPSAFVVKQPGKEITAKEVYDYLAERVSHTKYLRGGVRF
VDSIPRNVTGKITRKELLKQLLEKAGG-STOP).
SEQ ID NO: 19 is Signal peptide + FLAG tag-DOR-Gly-Ser-CBGct
(395-542)-STOP
(MKTIIALSYIFCLVFADYKDDDDA-
MELVPSARAELQSSPLVNLSDAFPSAFPSAGANASGSPGARSASSLALAIAITALYSAVCAV
GLLGNVLVMFGIVRYTKLKTATNIYIFNLALADALATSTLPFQSAKYLMETWPFGELLCKA
VLSIDYYNMFTSIFTLTMMSVDRYIAVCHPVKALDFRTPAKAKLINICIWVLASGVGVPIMV
MAVTQPRDGAVVCMLQFPSPSWYWDTVTKICVFLFAFVVPILIITVCYGLMLLRLRSVRLL
SGSKEKDRSLRRITRMVLVVVGAFVVCWAPIHIFVIVWTLVDINRRDPLVVAALHLCIALG
YANSSLNPVLYAFLDENFKRCFRQLCRTPCGRQEPGSLRRPRQATTRERVTACTPSDGPGG
GAAA-GS-
SKGYVNNVEATKEAIDDDGWLHSGDFGYYDEDEEIFYVVDRYKELIKYKGSQVAPAELEEI
LLKNPCIRDVAVVGIPDLEAGELPSAFVVKQPGKEITAKEVYDYLAERVSHTKYLRGGVRF
VDSIPRNVTGKITRKELLKQLLEKAGG-STOP).
SEQ ID NO: 20 is CBGnt (1-413)-Ser-Gly-Leu-Lys-Ser-Arg-Arg-Ala-
Leu-Asp-Ser-Ala-Barr1
(MVKREKNVIYGPEPLHPLEDLTAGEMLFRALRKHSHLPQALVDVVGDESLSYKEFFEATV
LLAQSLHNCGYKMNDVVSICAENNTRFFIPVIAAWYIGMIVAPVNESYIPDELCKVMGISKP
QIVFTTKNILNKVLEVQSRTNFIKRIIILDTVENIHGCESLPNFISRYSDGNIANFKPLHFDPVE
QVAAILCSSGTTGLPKGVMQTHQNICVRLIHALDPRVGTQLIPGVTVLVYLPFFHAFGFSITL
GYFMVGLRVIMFRRFDQEAFLKAIQDYEVRSVINVPSVILFLSKSPLVDKYDLSSLRELCCG
AAPLAKEVAEVAAKRLNLPGIRCGFGLTESTSANIHSLGDEFKSGSLGRVTPLMAAKIADRE
TGKALGPNQVGELCIKGPMVSKGYVNNVEATKEAIDDDG-SGLKSRRALDSA-
GDKGTRVFKKASPNGKLTVYLGKRDFVDHIDLVDPVDGVVLVDPEYLKERRVYVTLTCAF
RYGREDLDVLGLTFRKDLFVANVQSFPPAPEDKKPLTRLQERLIKKLGEHAYPFTFEIPPNL
PCSVTLQPGPEDTGKACGVDYEVKAFCAENLEEKIHKRNSVRLVIRKVQYAPERPGPQPTA
ETTRQFLMSDKPLHLEASLDKEIYYHGEPISVNVHVTNNTNKTVKKIKISVRQYADICLFNT
AQYKCPVAMEEADDTVAPSSTFCKVYTLTPFLANNREKRGLALDGKLKHEDTNLASSTLL
REGANREILGIIVSYKVKVKLVVSRGGDVAVELPFTLMHPKPKEEPPHREVPENETPVDTNL
IELDTNDDDIVFEDFARQRLKGMKDDKEEEEDGTGSPQLNNR-STOP).
SEQ ID NO: 21 is
(MVKREKNVIYGPEPLHPLEDLTAGEMLFRALRKHSHLPQALVDVVGDESLSYKEFFEATV
LLAQSLHNCGYKMNDVVSICAENNTRFFIPVIAAWYIGMIVAPVNESYIPDELCKVMGISKP
QIVFTTKNILNKVLEVQSRTNFIKRIIILDTVENIHGCESLPNFISRYSDGNIANFKPLHFDPVE
QVAAILCSSGTTGLPKGVMQTHQNICVRLIHALDPRYGTQLIPGVTVLVYLPFFHAFGFHIT
LGYFMVGLRVIMFRRFDQEAFLKAIQDYEVRSVINVPSVILFLSKSPLVDKYDLSSLRELCC
GAAPLAKEVAEVAAKRLNLPGIRCGFGLTESTSAIIQTLGDEFKSGSLGRVTPLMAAKIADR
ETGKALGPNQVGELCIKGPMVSKGYVNNVEATKEAIDDDG-SGLKSRRALDSA-
GEKPGTRVFKKSSPNCKLTVYLGKRDFVDHLDKVDPVDGVVLVDPDYLKDRKVFVTLTC
AFRYGREDLDVLGLSFRKDLFIATYQAFPPVPNPPRPPTRLQDRLLRKLGQHARPFFFTIPQN
LPCSVTLQPGPEDTGKACGVDFEIRAFCAKSLEEKSHKRNSVRLVIRKVQFAPEKPGPQPSA
ETTRHFLMSDRSLHLEASLDKELYYHGEPLNVNVHVTNNSTKTVKKIKVSVRQYADICLFS
TAQYKCPVAQLEQDDQVSPSSTFCKVYTITPLLSDNREKRGLALDGKLKHEDTNLASSTIV
KEGANKEVLGILVSYRVKVKLVVSRGGDVSVELPFVLMHPKPHDHIPLPRPQSAAPETDVP
VDTNLIEFDTNYATDDDIVFEDFARLRLKGMKDDDYDDQLC-STOP).
SEQ ID NO: 22 is |NotI|-DOR (338-372)-G1y-Ser-CBGet (395-542)-
STOP-|PasI|:
GRQEPGSLRRPRQATTRERVTACTPSDGPGGGAAA-GS-
SKGYVNNVEATKEAIDDDGWLHSGDFGYYDEDEHFYVVDRYKELIKYKGSQVAP
AELEEILLKNPCIRDVAVVGIPDLEAGELPSAFVVKQPGKEITAKEVYDYLAERVS
HTKYLRGGVRFVDSIPRNVTGKITRKELLKQLLEKAGG-STOP-|PasI|.
SEQ ID NO: 23 is |NheI|-CBGnt (1-413)-Ser-Gly-Leu-Lys-Ser-Arg-Arg-
Ala-Leu-Asp-Ser-Ala-Barr1 (2-169)-|BspEI|(|NheI|-
MVKREKNVIYGPEPLHPLEDLTAGEMLFRALRKHSHLPQALVDVVGDESLSYKEFFEATVL
LAQSLHNCGYKMNDVVSICAENNTRFFIPVIAAWYIGMIVAPVNESYIPDELCKVMGISKPQ
IVFTTKNILNKVLEVQSRTNFIKRIIILDTVENIHGCESLPNFISRYSDGNIANFKPLHFDPVEQ
VAAILCSSGTTGLPKGVMQTHQNICVRLIHALDPRVGTQLIPGVTVLVYLPFFHAFGFSITLG
YFMVGLRVIMFRRFDQEAFLKAIQDYEVRSVINVPSVILFLSKSPLVDKYDLSSLRELCCGA
APLAKEVAEVAAKRLNLPGIRCGFGLTESTSANIHSLGDEFKSGSLGRVTPLMAAKIADRET
GKALGPNQVGELCIKGPMVSKGYVNNVEATKEAIDDDG-SGLKSRRALDSA-
GDKGTRVFKKASPNGKLTVYLGKRDFVDHIDLVDPVDGVVLVDPEYLKERRVYVTLTCAF
RYGREDLDVLGLTFRKDLFVANVQSFPPAPEDKKPLTRLQERLIKKLGEHAYPFTFEIPPNL
PCSVTLQPGPEDTGKACGVDYEVKAFCAENLEEKIHKRNSVRLVIR-|BspEI|)).
SEQ ID NO: 24 is |NheI|-CBRnt (1-413)-Ser-Gly-Leu-Lys-Ser-Arg-Arg-
Ala-Leu-Asp-Ser-Ala-Barr2 (2-196)-|AgeI|(|NheI|-
MVKREKNVIYGPEPLHPLEDLTAGEMLFRALRKHSHLPQALVDVVGDESLSYKEFFEATVL
LAQSLHNCGYKMNDVVSICAENNTRFFIPVIAAWYIGMIVAPVNESYIPDELCKVMGISKPQ
IVFTTKNILNKVLEVQSRTNFIKRIIILDTVENIHGCESLPNFISRYSDGNIANFKPLHFDPVEQ
VAAILCSSGTTGLPKGVMQTHQNICVRLIHALDPRYGTQLIPGVTVLVYLPFFHAFGFHITL
GYFMVGLRVIMFRRFDQEAFLKAIQDYEVRSVINVPSVILFLSKSPLVDKYDLSSLRELCCG
AAPLAKEVAEVAAKRLNLPGIRCGFGLTESTSAIIQTLGDEFKSGSLGRVTPLMAAKIADRE
TGKALGPNQVGELCIKGPMVSKGYVNNVEATKEAIDDDG-SGLKSRRALDSA-
GEKPGTRVFKKSSPNCKLTVYLGKRDFVDHLDKVDPVDGVVLVDPDYLKDRKVFVTLTC
AFRYGREDLDVLGLSFRKDLFIATYQAFPPVPNPPRPPTRLQDRLLRKLGQHMAPFFFTIPQN
LPCSVTLQPGPEDTGKACGVDFEIRAFCAKSLEEKSHKRNSVRLVIRKVQFAPEKPGPQPSA
ETTRHFLMSDR-|AgeI|).
SEQ ID NO: 25 is Signal peptide + FLAG tag-DOR-
GSSGGGGSGGGGSGGGGSGGGG-CB Gct(395-542):
MKTIIALSYIFCLVFADYKDDDDA-
MELVPSARAELQSSPLVNLSDAFPSAFPSAGANASGSPGARSASSLALAIAITALYSAVCAV
GLLGNVLVMFGIVRYTKLKTATNIYIFNLALADALATSTLPFQSAKYLMETWPFGELLCKA
VLSIDYYNMFTSIFTLTMMSVDRYIAVCHPVKALDFRTPAKAKLINICIWVLASGVGVPIMV
MAVTQPRDGAVVCMLQFPSPSWYWDTVTKICVFLFAFVVPILIITVCYGLMLLRLRSVRLL
SGSKEKDRSLRRITRMVLVVVGAFVVCWAPIHIFVIVWTLVDINRRDPLVVAALHLCIALG
YANSSLNPVLYAFLDENFKRCFRQLCRTPCGRQEPGSLRRPRQATTRERVTACTPSDGPGG
GAAA-GSSGGGGSGGGGSGGGGSGGGG-
SKGYVNNVEATKEAIDDDGWLHSGDFGYYDEDEHFYVVDRYKELIKYKGSQVAPAELEEI
LLKNPCIRDVAVVGIPDLEAGELPSAFVVKQPGKEITAKEVYDYLAERVSHTKYLRGGVRF
VDSIPRNVTGKITRKELLKQLLEKAGG-STOP.
SEQ ID NO: 26 is CBGnt(1-415)-SGGGGSGGGGSGGGGSGGGGS-Barr1:
MVKREKNVIYGPEPLEPLEDLTAGEMLFRALRKHSHLPQALVDVVGDESLSYKEFFEATVL
LAQSLHNCGYKMNDVVSICAENNTRFFIPVIAAWYIGMIVAPVNESYIPDELCKVMGISKPQ
IVFTTKNILNKVLEVQSRTNFIKRIIILDTVENIHGCESLPNFISRYSDGNIANFKPLHFDPVEQ
VAAILCSSGTTGLPKGVMQTHQNICVRLIHALDPRVGTQLIPGVTVLVYLPFFHAFGFSITLG
YFMVGLRVIMFRRFDQEAFLKAIQDYEVRSVINVPSVILFLSKSPLVDKYDLSSLRELCCGA
APLAKEVAEVAAKRLNLPGIRCGFGLTESTSANIHSLGDEFKSGSLGRVTPLMAAKIADRET
GKALGPNQVGELCIKGPMVSKGYVNNVEATKEAIDDDGWL-
SGGGGSGGGGSGGGGSGGGGS-
GDKGTRVFKKASPNGKLTVYLGKRDFVDHIDLVDPVDGVVLVDPEYLKERRVYVTLTCAF
RYGREDLDVLGLTFRKDLFVANVQSFPPAPEDKKPLTRLQERLIKKLGEHAYPFTFEIPPNL
PCSVTLQPGPEDTGKACGVDYEVKAFCAENLEEKIHKRNSVRLVIRKVQYAPERPGPQPTA
ETTRQFLMSDKPLHLEASLDKEIYYHGEPISVNVHVTNNTNKTVKKIKISVRQYADICLFNT
AQYKCPVAMEEADDTVAPSSTFCKVYTLTPFLANNREKRGLALDGKLKHEDTNLASSTLL
REGANREILGIIVSYKVKVKLVVSRGGDVAVELPFTLMHPKPKEEPPHREVPENETPVDTNL
IELDTNDDDIVFEDFARQRLKGMKDDKEEEEDGTGSPQLNNR-STOP.
SEQ ID NO: 27 is CBRnt(1-413)-SGGGGSGGGGS-Barr2:
MVKREKNVIYGPEPLEHPLEDLTAGEMLFRALRKHSHLPQALVDVVGDESLSYKEFFEATVL
LAQSLHNCGYKMNDVVSICAENNTRFFIPVIAAWYIGMIVAPVNESYIPDELCKVMGISKPQ
IVFTTKNILNKVLEVQSRTNFIKRIIILDTVENIHGCESLPNFISRYSDGNIANFKPLHFDPVEQ
VAAILCSSGTTGLPKGVMQTHQNICVRLIHALDPRYGTQLIPGVTVLVYLPFFHAFGFHITL
GYFMVGLRVEVIMFRRFDQEAFLKAIQDYEVRSVINVPSVILFLSKSPLVDKYDLSSLRELCCG
AAPLAKEVAEVAAKRLNLPGIRCGFGLTESTSAIIQTLGDEFKSGSLGRVTPLMAAKIADRE
TGKALGPNQVGELCIKGPMVSKGYVNNVEATKEAIDDDG-SGGGGSGGGGS-
GEKPGTRVFKKSSPNCKLTVYLGKRDFVDHLDKVDPVDGVVLVDPDYLKDRKVFVTLTC
AFRYGREDLDVLGLSFRKDLFIATYQAFPPVPNPPRPPTRLQDRLLRKLGQHAHPFFFTIPQN
LPCSVTLQPGPEDTGKACGVDFEIRAFCAKSLEEKSHKRNSVRLVIRKVQFAPEKPGPQPSA
ETTRHFLMSDRSLHLEASLDKELYYHGEPLNVNVHVTNNSTKTVKKIKVSVRQYADICLFS
TAQYKCPVAQLEQDDQVSPSSTFCKVYTITPLLSDNREKRGLALDGKLKHEDTNLASSTIV
KEGANKEVLGILVSYRVKVKLVVSRGGDVSVELPFVLMHPKPHDHIPLPRPQSAAPETDVP
VDTNLIEFDTNYATDDDIVFEDFARLRLKGMKDDDYDDQLC-STOP.
SEQ ID NO: 28 is a Signal peptide + FLAG tag-KOR-
GSSGGGGSGGGGSGGGGSGGGG-CB Gct(395-542):
MKTIIALSYIFCLVFADYKDDDDA-
MESPIQIFRGDPGPTCSPSACLLPNSSSWFPNWAESDSNGSVGSEDQQLESAHISPAIPVIITA
VYSVVFVVGLVGNSLVMFVIIRYTKMKTATNIYIFNLALADALVTTTMPFQSAVYLMNSW
PFGDVLCKIVISIDYYNMFTSIFTLTMMSVDRYIAVCHPVKALDFRTPLKAKIINICIWLLASS
VGISAIVLGGTKVREDVDVIECSLQFPDDEYSWWDLFMKICVFVFAFVIPVLIIIVCYTLM1L
RLKSVRLLSGSREKDRNLRRITKLVLVVVAVFIICWTPIHIFILVEALGSTSHSTAALSSYYFC
IALGYTNSSLNPVLYAFLDENFKRCFRDFCFPIKMRMERQSTNRVRNTVQDPASMRDVGG
MNKPV-GSSGGGGSGGGGSGGGGSGGGG-
SKGYVNNVEATKEAIDDDGWLHSGDFGYYDEDEHFYVVDRYKELIKYKGSQVAPAELEEI
LLKNPCIRDVAVVGIPDLEAGELPSAFVVKQPGKEITAKEVYDYLAERVSHTKYLRGGVRF
VDSIPRNVTGKITRKELLKQLLEKAGG-STOP.
SEQ ID NO: 29 is D2R-GSSGGGGSGGGGSGGGGSGGGG-CBGct(395-542):
MDPLNLSWYDDDLERQNWSRPFNGSDGKADRPHYNYYATLLTLLIAVIVFGNVLVCMAV
SREKALQTTTNYLIVSLAVADLLVATLVMPWVVYLEVVGEWKFSRIFICDIFVTLDVMMCT
ASILNLCAISIDRYTAVAMPMLYNTRYSSKRRVTVMISIVWVLSFTISCPLLFGLNNADQNE
CIIANPAFVVYSSIVSFYVPFIVTLLVYIKIYIVLRRRRKRVNTKRSSRAFRAHLRAPLKGNCT
HPEDMKLCTVIMKSNGSFPVNRRRVEAARRAQELEMEMLSSTSPPERTRYSPIPPSHHQLTL
PDPSHHGLHSTPDSPAKPEKNGHAKDEEPKIAKIFEIQTMPNGKTRTSLKTMSRRKLSQQKE
KKATQMLAIVLGVFIICWLPFFITHILNIHCDCNIPPVLYSAFTWLGYVNSAVNPIIYTTFNIEF
RKAFLKILHC-GSSGGGGSGGGGSGGGGSGGGG-
SKGYVNNVEATKEAIDDDGWLHSGDFGYYDEDEHFYVVDRYKELIKYKGSQVAPAELEEI
LLKNPCIRDVAVVGIPDLEAGELPSAFVVKQPGKEITAKEVYDYLAERVSHTKYLRGGVRF
VDSIPRNVTGKITRKELLKQLLEKAGG-STOP.
SEQ ID NO: 30 is 3xHA-AGTR1(2-360)-GSSGGGGSGGGGSGGGGSGGGG-
CB Gct(395-542):
MYPYDVPDYAYPYDVPDYAYPYDVPDYA-
DILNSSTEDGIKRIQDDCPKAGRHNYIFVMIPTLYSIIFVVGIFGNSLVVIVIYFYMKLKTVAS
VFLLNLALADLCFLLTLPLWAVYTAMEYRWPFGNYLCKIASASVSFNLYASVFLLTCLSID
RYLAIVHPMKSRLRRTMLVAKVTCIIIWLLAGLASLPAIIHRNVFFIENTNITVCAFHYESQN
STLPIGLGLTKNILGFLFPFLIILTSYTLIWKALKKAYEIQKNKPRNDDIFKIIMAIVLFFFFSWI
PHQIFTFLDVLIQLGIIRDCRIADIVDTAMPITICIAYFNNCLNPLFYGFLGKKFKRYFLQLLK
YIPPKAKSHSNLSTKMSTLSYRPSDNVSSSTKKPAPCFEVE-
GSSGGGGSGGGGSGGGGSGGGG-
SKGYVNNVEATKEAIDDDGWLHSGDFGYYDEDDIFYVVDRYKELIKYKGSQVAPAELEEI
LLKNPCIRDVAVVGIPDLEAGELPSAFVVKQPGKEITAKEVYDYLAERVSHTKYLRGGVRF
VDSIPRNVTGKITRKELLKQLLEKAGG-STOP.

What is claimed is:

1. A method of determining if a test agent preferentially recruits an isoform of β-arrestin to a G protein-coupled receptor (GPCR), which method comprises:
(i) contacting a cell, which expresses:
(a) a GPCR linked to a fragment of an enzyme, which, in the presence of a substrate for the enzyme, produces an optically detectable signal upon refolding with a complementary fragment of the enzyme linked to an isoform of β-arrestin,
(b) a first isoform of β-arrestin linked to a first complementary fragment of the enzyme, which produces an optically detectable signal of a first λₘₐₓ (i.e., peak emission wavelength) upon refolding with the complementary fragment of the enzyme linked to the GPCR and,
(c) a second isoform of β-arrestin linked to a second complementary fragment of the enzyme, which produces an optically detectable signal of a second λₘₐₓ upon refolding with the complementary fragment of the enzyme linked to the GPCR,
with:
(a) a substrate for the enzyme, and
(b) the test agent; and
(ii) detecting, and optionally quantitating, the optically detectable signal of the first λₘₐₓ and the optically detectable signal of the second λₘₐₓ;
wherein an absolute value of a ratio of the amount of signal of the first λₘₐₓ to the amount of signal of the second λₘₐₓ is compared to an absolute value of a corresponding ratio obtained for a reference agent and indicates whether the agent preferentially recruits an isoform of β-arrestin to the receptor.

2. The method of claim 1, wherein the test agent is an agonist, an antagonist, a sensitizing agent, or a desensitizing agent.

3. The method of claim 1, wherein the enzyme is a luciferase and the substrate for the enzyme is a luciferin.

4. The method of claim 3, wherein the luciferase is a click beetle luciferase.

5. The method of claim 1, wherein the fragment of the enzyme linked to the GPCR is a C-terminal fragment.

6. The method of claim 5, wherein the C-terminal fragment is a C-terminal fragment of click beetle green (CBG) luciferase.

7. The method of claim 5, wherein the first complementary fragment of the enzyme linked to the first isoform of β-arrestin and the second complementary fragment of the enzyme linked to the second isoform of β-arrestin are N-terminal fragments.

8. The method of claim 7, wherein the first complementary fragment of the enzyme linked to the first isoform of β-arrestin is an N-terminal fragment from a CBG luciferase.

9. The method of claim 7, wherein the second complementary fragment of the enzyme linked to the second isoform of β-arrestin is an N-terminal fragment from a click beetle red (CBR) luciferase.

10. The method of claim 1, wherein the GPCR is δ opioid receptor (DOR), κ opioid receptor (KOR), dopamine receptor, or angiotensin receptor.

11. The method of claim 1, wherein the first isoform of β-arrestin is β-arrestin 1 (Barr1).

12. The method ofclaim 1, wherein the second isoform of β-arrestin is β-arrestin 2 (Barr2).

13. The method of claim 1, wherein the cell is in a well on a substrate.

14. The method of claim 1, wherein the cell is in a living animal.

15. The method of claim 1, wherein a cell lysate preparation is used in place of the cell.

16. A biosensor comprising:
(i) (a) a cell, which expresses a G protein-coupled receptor (GPCR) linked to a fragment of an enzyme, which, in the presence of a substrate for the enzyme, produces an optically detectable signal upon refolding with a complementary fragment of the enzyme linked to an isoform of β-arrestin, or (b) a cell lysate prepared from (a), and
(ii) (a) a first isoform of β-arrestin linked to a first complementary fragment of the enzyme, which produces an optically detectable signal of a first λₘₐₓ upon refolding with the complementary fragment of the enzyme linked to the GPCR, and
(b) a second isoform of β-arrestin linked to a second complementary fragment of the enzyme, which produces an optically detectable signal of a second λₘₐₓ upon refolding with the complementary fragment of the enzyme linked to the GPCR.

17. The biosensor of claim 16, which further comprises (iii) a substrate for the enzyme.

18. The biosensor of claim 17, wherein the enzyme is luciferase and the substrate is a luciferin.

19. The biosensor of claim 18, wherein the luciferase is a click beetle luciferase.

20. The biosensor of claim 16, wherein the fragment of the enzyme linked to the GPCR is a C-terminal fragment.

21. The biosensor of claim 20, wherein the C-terminal fragment is a C-terminal fragment of click beetle green (CBG) luciferase.

22. The biosensor of claim 20, wherein the first complementary fragment of the enzyme linked to the first isoform of β-arrestin and the second complementary fragment of the enzyme linked to the second isoform of β-arrestin are N-terminal fragments.

23. The biosensor of claim 22, wherein the first complementary fragment of the enzyme linked to the first isoform of β-arrestin is an N-terminal fragment from a click beetle green (CBG) luciferase.

24. The biosensor of claim 22, wherein the second complementary fragment of the enzyme linked to the second isoform of β-arrestin is an N-terminal fragment from a click beetle red (CBR) luciferase.

25. The biosensor of claim 16, wherein the GPCR is δ opioid receptor (DOR), κ opioid receptor (KOR), dopamine receptor, or angiotensin receptor.

26. The biosensor of claim 16, wherein the first isoform of β-arrestin is β-arrestin 1 (Barr1).

27. The biosensor of claim 16, wherein the second isoform of β-arrestin is β-arrestin 2 (Barr2).

28. The biosensor of claim 16, wherein the cell or cell lysate is in a well on a substrate.

29. A bioarray comprising biosensors of claim 16.

30. A kit comprising (a) the biosensor of claim 16 or a bioarray of the biosensors of claim 16 and (b) instructions for determining if an agent preferentially recruits an isoform of β-arrestin to a GPCR.


SEQUENCE LISTING (30 sequences)

SEQ ID NO: 1 - protein, 7 aa, Artificial Sequence
  Synthetic construct
GSSGGGG

SEQ ID NO: 2 - protein, 12 aa, Artificial Sequence
  Synthetic construct
GSSGGGGSGGGG

SEQ ID NO: 3 - protein, 17 aa, Artificial Sequence
  Synthetic construct
GSSGGGGSGGGGSGGGG

SEQ ID NO: 4 - protein, 22 aa, Artificial Sequence
  Synthetic construct
GSSGGGGSGGGGSGGGGSGGGG

SEQ ID NO: 5 - protein, 27 aa, Artificial Sequence
  Synthetic construct
GSSGGGGSGGGGSGGGGSGGGGSGGGG

SEQ ID NO: 6 - protein, 12 aa, Artificial Sequence
  Synthetic construct
SGLKSRRALDSA

SEQ ID NO: 7 - protein, 6 aa, Artificial Sequence
  Synthetic construct
GGGGGS

SEQ ID NO: 8 - protein, 11 aa, Artificial Sequence
  Synthetic construct
SGGGGSGGGGS

SEQ ID NO: 9 - protein, 16 aa, Artificial Sequence
  Synthetic construct
SGGGGSGGGGSGGGGS

SEQ ID NO: 10 - protein, 21 aa, Artificial Sequence
  Synthetic construct
SGGGGSGGGGSGGGGSGGGGS

SEQ ID NO: 11 - protein, 26 aa, Artificial Sequence
  Synthetic construct
SGGGGSGGGGSGGGGSGGGGSGGGGS

SEQ ID NO: 12 - DNA, 567 bp, Artificial Sequence
  Synthetic construct of CBGct ligation fragment which encodes SEQ ID NO 22
gcggccgccaagaacccggcagtctccgtcgtccccgccaggccaccacgcgtgagcgtgtcactgcctgcaccccctccgacggcccgggcggtggcgccgccgccggcagtagcaagggttatgtcaataacgttgaagctaccaaggaggccatcgacgacgacggctggttgcattctggtgattttggatattacgacgaagatgagcatttttacgtcgtggatcgttacaaggagctgatcaaatacaagggtagccaggttgctccagctgagttggaggagattctgttgaaaaatccatgcattcgcgatgtcgctgtggtcggcattcctgatctggaggccggcgaactgccttctgctttcgttgtcaagcagcctggtaaagaaattaccgccaaagaagtgtatgattacctggctgaacgtgtgagccatactaagtacttgcgtggcggcgtgcgttttgttgactccatccctcgtaacgtaacaggcaaaattacccgcaaggagctgttgaaacaattgttggagaaggccggcggttgaccctggg

SEQ ID NO: 13 - DNA, 1795 bp, Artificial Sequence
  Synthetic construct of CBGnt ligation fragment which encodes SEQ ID NO 23
gctagcgccgccaccatggtgaagcgtgagaaaaatgtcatctatggccctgagcctctccatcctttggaggatttgactgccggcgaaatgctgtttcgtgctctccgcaagcactctcatttgcctcaagccttggtcgatgtggtcggcgatgaatctttgagctacaaggagttttttgaggcaaccgtcttgctggctcagtccctccacaattgtggctacaagatgaacgacgtcgttagtatctgtgctgaaaacaatacccgtttcttcattccagtcatcgccgcatggtatatcggtatgatcgtggctccagtcaacgagagctacattcccgacgaactgtgtaaagtcatgggtatctctaagccacagattgtcttcaccactaagaatattctgaacaaagtcctggaagtccaaagccgcaccaactttattaagcgtatcatcatcttggacactgtggagaatattcacggttgcgaatctttgcctaatttcatctctcgctattcagacggcaacatcgcaaactttaaaccactccacttcgaccctgtggaacaagttgcagccattctgtgtagcagcggtactactggactcccaaagggagtcatgcagacccatcaaaacatttgcgtgcgtctgatccatgctctcgatccacgcgtgggcactcagctgattcctggtgtcaccgtcttggtctacttgcctttcttccatgctttcggctttagcattactttgggttactttatggtcggtctccgcgtgattatgttccgccgttttgatcaggaggctttcttgaaagccatccaagattatgaagtccgcagtgtcatcaacgtgcctagcgtgatcctgtttttgtctaagagcccactcgtggacaagtacgacttgtcttcactgcgtgaattgtgttgcggtgccgctccactggctaaggaggtcgctgaagtggccgccaaacgcttgaatcttccagggattcgttgtggcttcggcctcaccgaatctaccagcgcaaacattcactctctcggggatgagtttaagagcggctctttgggccgtgtcactccactcatggctgctaagatcgctgatcgcgaaactggtaaggctttgggcccgaaccaagtgggcgagctgtgtatcaaaggccctatggtgagcaagggttatgtcaataacgttgaagctaccaaggaggccatcgacgacgacggcagcggcctgaagagcaggagggccctggacagcgctggcgacaaagggacccgagtgttcaagaaggccagtccaaatggaaagctcaccgtctacctgggaaagcgggactttgtggaccacatcgacctcgtggaccctgtggatggtgtggtcctggtcgatcctgagtatctcaaagagcggagagtctatgtgacgctgacctgcgccttccgctatggccgggaggacctggatgtcctgggcctgacctttcgcaaggacctgtttgtggccaacgtacagtcgttcccaccggcccccgaggacaagaagcccctgacgcggctgcaggaacgcctcatcaagaagctgggcgagcacgcttaccctttcacctttgagatccctccaaaccttccatgttctgtgacactgcagccggggcccgaagacacggggaaggcttgcggtgtggactatgaagtcaaagccttctgcgcggagaatttggaggagaagatccacaagcgcaattctgtgcgtctggtcatccgga

SEQ ID NO: 14 - DNA, 1876 bp, Artificial Sequence
  Synthetic construct for CBRnt ligation fragment which encodes SEQ ID NO 24
gctagcgccgccaccatggtaaagcgtgagaaaaatgtcatctatggccctgagcctctccatcctttggaggatttgactgccggcgaaatgctgtttcgtgctctccgcaagcactctcatttgcctcaagccttggtcgatgtggtcggcgatgaatctttgagctacaaggagttttttgaggcaaccgtcttgctggctcagtccctccacaattgtggctacaagatgaacgacgtcgttagtatctgtgctgaaaacaatacccgtttcttcattccagtcatcgccgcatggtatatcggtatgatcgtggctccagtcaacgagagctacattcccgacgaactgtgtaaagtcatgggtatctctaagccacagattgtcttcaccactaagaatattctgaacaaagtcctggaagtccaaagccgcaccaactttattaagcgtatcatcatcttggacactgtggagaatattcacggttgcgaatctttgcctaatttcatctctcgctattcagacggcaacatcgcaaactttaaaccactccacttcgaccctgtggaacaagttgcagccattctgtgtagcagcggtactactggactcccaaagggagtcatgcagacccatcaaaacatttgcgtgcgtctgatccatgctctcgatccacgctacggcactcagctgattcctggtgtcaccgtcttggtctacttgcctttcttccatgctttcggctttcatattactttgggttactttatggtcggtctccgcgtgattatgttccgccgttttgatcaggaggctttcttgaaagccatccaagattatgaagtccgcagtgtcatcaacgtgcctagcgtgatcctgtttttgtctaagagcccactcgtggacaagtacgacttgtcttcactgcgtgaattgtgttgcggtgccgctccactggctaaggaggtcgctgaagtggccgccaaacgcttgaatcttccagggattcgttgtggcttcggcctcaccgaatctaccagtgcgattatccagactctcggggatgagtttaagagcggctctttgggccgtgtcactccactcatggctgctaagatcgctgatcgcgaaactggtaaggctttgggcccgaaccaagtgggcgagctgtgtatcaaaggccctatggtgagcaagggttatgtcaataacgttgaagctaccaaggaggccatcgacgacgacggcagcggcctgaagagcaggagggccctggacagcgctggggagaaacccgggaccagggtcttcaagaagtcgagccctaactgcaagctcaccgtgtacttgggcaagcgggacttcgtagatcacctggacaaagtggaccctgtagatggcgtggtgcttgtggaccctgactacctgaaggaccgcaaagtgtttgtgaccctcacctgcgccttccgctatggccgtgaagacctggatgtgctgggcttgtccttccgcaaagacctgttcatcgccacctaccaggccttccccccggtgcccaacccaccccggccccccacccgcctgcaggaccggctgctgaggaagctgggccagcatgcccaccccttcttcttcaccataccccagaatcttccatgctccgtcacactgcagccaggcccagaggatacaggaaaggcctgcggcgtagactttgagattcgagccttctgtgctaaatcactagaagagaaaagccacaaaaggaactctgtgcggctggtgatccgaaaggtgcagttcgccccggagaaacccggcccccagccttcagccgaaaccacacgccacttcctcatgtctgaccggt

SEQ ID NO: 15 - protein, 5 aa, Homo sapiens
YGGFL

SEQ ID NO: 16 - protein, 17 aa, Homo sapiens
YGGFLRRIRPKLKWDNQ

SEQ ID NO: 17 - protein, 8 aa, Homo sapiens
DRVYIHPF

SEQ ID NO: 18 - protein, 170 aa, Artificial Sequence
  Synthetic construct
GSSGGGGSGGGGSGGGGSGGGGSKGYVNNVEATKEAIDDDGWLHSGDFGYYDEDEHFYVVDRYKELIKYKGSQVAPAELEEILLKNPCIRDVAVVGIPDLEAGELPSAFVVKQPGKEITAKEVYDYLAERVSHTKYLRGGVRFVDSIPRNVTGKITRKELLKQLLEKAGG

SEQ ID NO: 19 - protein, 546 aa, Artificial Sequence
  Synthetic DOR-CBGct ligation product
MKTIIALSYIFCLVFADYKDDDDAMELVPSARAELQSSPLVNLSDAFPSAFPSAGANASGSPGARSASSLALAIAITALYSAVCAVGLLGNVLVMFGIVRYTKLKTATNIYIFNLALADALATSTLPFQSAKYLMETWPFGELLCKAVLSIDYYNMFTSIFTLTMMSVDRYIAVCHPVKALDFRTPAKAKLINICIWVLASGVGVPIMVMAVTQPRDGAVVCMLQFPSPSWYWDTVTKICVFLFAFVVPILIITVCYGLMLLRLRSVRLLSGSKEKDRSLRRITRMVLVVVGAFVVCWAPIHIFVIVWTLVDINRRDPLVVAALHLCIALGYANSSLNPVLYAFLDENFKRCFRQLCRTPCGRQEPGSLRRPRQATTRERVTACTPSDGPGGGAAAGSSKGYVNNVEATKEAIDDDGWLHSGDFGYYDEDEHFYVVDRYKELIKYKGSQVAPAELEEILLKNPCIRDVAVVGIPDLEAGELPSAFVVKQPGKEITAKEVYDYLAERVSHTKYLRGGVRFVDSIPRNVTGKITRKELLKQLLEKAGG

SEQ ID NO: 20 - protein, 834 aa, Artificial Sequence
  Synthetic CBGnt-Barr1 ligation product
MVKREKNVIYGPEPLHPLEDLTAGEMLFRALRKHSHLPQALVDVVGDESLSYKEFFEATVLLAQSLHNCGYKMNDVVSICAENNTRFFIPVIAAWYIGMIVAPVNESYIPDELCKVMGISKPQIVFTTKNILNKVLEVQSRTNFIKRIIILDTVENIHGCESLPNFISRYSDGNIANFKPLHFDPVEQVAAILCSSGTTGLPKGVMQTHQNICVRLIHALDPRVGTQLIPGVTVLVYLPFFHAFGFSITLGYFMVGLRVIMFRRFDQEAFLKAIQDYEVRSVINVPSVILFLSKSPLVDKYDLSSLRELCCGAAPLAKEVAEVAAKRLNLPGIRCGFGLTESTSANIHSLGDEFKSGSLGRVTPLMAAKIADRETGKALGPNQVGELCIKGPMVSKGYVNNVEATKEAIDDDGSGLKSRRALDSAGDKGTRVFKKASPNGKLTVYLGKRDFVDHIDLVDPVDGVVLVDPEYLKERRVYVTLTCAFRYGREDLDVLGLTFRKDLFVANVQSFPPAPEDKKPLTRLQERLIKKLGEHAYPFTFEIPPNLPCSVTLQPGPEDTGKACGVDYEVKAFCAENLEEKIHKRNSVRLVIRKVQYAPERPGPQPTAETTRQFLMSDKPLHLEASLDKEIYYHGEPISVNVHVTNNTNKTVKKIKISVRQYADICLFNTAQYKCPVAMEEADDTVAPSSTFCKVYTLTPFLANNREKRGLALDGKLKHEDTNLASSTLLREGANREILGIIVSYKVKVKLVVSRGGDVAVELPFTLMHPKPKEEPPHREVPENETPVDTNLIELDTNDDDIVFEDFARQRLKGMKDDKEEEEDGTGSPQLNNR

SEQ ID NO: 21 - protein, 833 aa, Artificial Sequence
  Synthetic CBRnt-Barr2 ligation product
MVKREKNVIYGPEPLHPLEDLTAGEMLFRALRKHSHLPQALVDVVGDESLSYKEFFEATVLLAQSLHNCGYKMNDVVSICAENNTRFFIPVIAAWYIGMIVAPVNESYIPDELCKVMGISKPQIVFTTKNILNKVLEVQSRTNFIKRIIILDTVENIHGCESLPNFISRYSDGNIANFKPLHFDPVEQVAAILCSSGTTGLPKGVMQTHQNICVRLIHALDPRYGTQLIPGVTVLVYLPFFHAFGFHITLGYFMVGLRVIMFRRFDQEAFLKAIQDYEVRSVINVPSVILFLSKSPLVDKYDLSSLRELCCGAAPLAKEVAEVAAKRLNLPGIRCGFGLTESTSAIIQTLGDEFKSGSLGRVTPLMAAKIADRETGKALGPNQVGELCIKGPMVSKGYVNNVEATKEAIDDDGSGLKSRRALDSAGEKPGTRVFKKSSPNCKLTVYLGKRDFVDHLDKVDPVDGVVLVDPDYLKDRKVFVTLTCAFRYGREDLDVLGLSFRKDLFIATYQAFPPVPNPPRPPTRLQDRLLRKLGQHAHPFFFTIPQNLPCSVTLQPGPEDTGKACGVDFEIRAFCAKSLEEKSHKRNSVRLVIRKVQFAPEKPGPQPSAETTRHFLMSDRSLHLEASLDKELYYHGEPLNVNVHVTNNSTKTVKKIKVSVRQYADICLFSTAQYKCPVAQLEQDDQVSPSSTFCKVYTITPLLSDNREKRGLALDGKLKHEDTNLASSTIVKEGANKEVLGILVSYRVKVKLVVSRGGDVSVELPFVLMHPKPHDHIPLPRPQSAAPETDVPVDTNLIEFDTNYATDDDIVFEDFARLRLKGMKDDDYDDQLC

SEQ ID NO: 22 - protein, 185 aa, Artificial Sequence
  Synthetic product
GRQEPGSLRRPRQATTRERVTACTPSDGPGGGAAAGSSKGYVNNVEATKEAIDDDGWLHSGDFGYYDEDEHFYVVDRYKELIKYKGSQVAPAELEEILLKNPCIRDVAVVGIPDLEAGELPSAFVVKQPGKEITAKEVYDYLAERVSHTKYLRGGVRFVDSIPRNVTGKITRKELLKQLLEKAGG

SEQ ID NO: 23 - protein, 593 aa, Artificial Sequence
  Synthetic product
MVKREKNVIYGPEPLHPLEDLTAGEMLFRALRKHSHLPQALVDVVGDESLSYKEFFEATVLLAQSLHNCGYKMNDVVSICAENNTRFFIPVIAAWYIGMIVAPVNESYIPDELCKVMGISKPQIVFTTKNILNKVLEVQSRTNFIKRIIILDTVENIHGCESLPNFISRYSDGNIANFKPLHFDPVEQVAAILCSSGTTGLPKGVMQTHQNICVRLIHALDPRVGTQLIPGVTVLVYLPFFHAFGFSITLGYFMVGLRVIMFRRFDQEAFLKAIQDYEVRSVINVPSVILFLSKSPLVDKYDLSSLRELCCGAAPLAKEVAEVAAKRLNLPGIRCGFGLTESTSANIHSLGDEFKSGSLGRVTPLMAAKIADRETGKALGPNQVGELCIKGPMVSKGYVNNVEATKEAIDDDGSGLKSRRALDSAGDKGTRVFKKASPNGKLTVYLGKRDFVDHIDLVDPVDGVVLVDPEYLKERRVYVTLTCAFRYGREDLDVLGLTFRKDLFVANVQSFPPAPEDKKPLTRLQERLIKKLGEHAYPFTFEIPPNLPCSVTLQPGPEDTGKACGVDYEVKAFCAENLEEKIHKRNSVRLVIR

SEQ ID NO: 24 - protein, 620 aa, Artificial Sequence
  Synthetic product
MVKREKNVIYGPEPLHPLEDLTAGEMLFRALRKHSHLPQALVDVVGDESLSYKEFFEATVLLAQSLHNCGYKMNDVVSICAENNTRFFIPVIAAWYIGMIVAPVNESYIPDELCKVMGISKPQIVFTTKNILNKVLEVQSRTNFIKRIIILDTVENIHGCESLPNFISRYSDGNIANFKPLHFDPVEQVAAILCSSGTTGLPKGVMQTHQNICVRLIHALDPRYGTQLIPGVTVLVYLPFFHAFGFHITLGYFMVGLRVIMFRRFDQEAFLKAIQDYEVRSVINVPSVILFLSKSPLVDKYDLSSLRELCCGAAPLAKEVAEVAAKRLNLPGIRCGFGLTESTSAIIQTLGDEFKSGSLGRVTPLMAAKIADRETGKALGPNQVGELCIKGPMVSKGYVNNVEATKEAIDDDGSGLKSRRALDSAGEKPGTRVFKKSSPNCKLTVYLGKRDFVDHLDKVDPVDGVVLVDPDYLKDRKVFVTLTCAFRYGREDLDVLGLSFRKDLFIATYQAFPPVPNPPRPPTRLQDRLLRKLGQHAHPFFFTIPQNLPCSVTLQPGPEDTGKACGVDFEIRAFCAKSLEEKSHKRNSVRLVIRKVQFAPEKPGPQPSAETTRHFLMSDR

SEQ ID NO: 25 - protein, 566 aa, Artificial Sequence
  Synthetic DOR construct
MKTIIALSYIFCLVFADYKDDDDAMELVPSARAELQSSPLVNLSDAFPSAFPSAGANASGSPGARSASSLALAIAITALYSAVCAVGLLGNVLVMFGIVRYTKLKTATNIYIFNLALADALATSTLPFQSAKYLMETWPFGELLCKAVLSIDYYNMFTSIFTLTMMSVDRYIAVCHPVKALDFRTPAKAKLINICIWVLASGVGVPIMVMAVTQPRDGAVVCMLQFPSPSWYWDTVTKICVFLFAFVVPILIITVCYGLMLLRLRSVRLLSGSKEKDRSLRRITRMVLVVVGAFVVCWAPIHIFVIVWTLVDINRRDPLVVAALHLCIALGYANSSLNPVLYAFLDENFKRCFRQLCRTPCGRQEPGSLRRPRQATTRERVTACTPSDGPGGGAAAGSSGGGGSGGGGSGGGGSGGGGSKGYVNNVEATKEAIDDDGWLHSGDFGYYDEDEHFYVVDRYKELIKYKGSQVAPAELEEILLKNPCIRDVAVVGIPDLEAGELPSAFVVKQPGKEITAKEVYDYLAERVSHTKYLRGGVRFVDSIPRNVTGKITRKELLKQLLEKAGG

SEQ ID NO: 26 - protein, 845 aa, Artificial Sequence
  Synthetic Barr1 construct
MVKREKNVIYGPEPLHPLEDLTAGEMLFRALRKHSHLPQALVDVVGDESLSYKEFFEATVLLAQSLHNCGYKMNDVVSICAENNTRFFIPVIAAWYIGMIVAPVNESYIPDELCKVMGISKPQIVFTTKNILNKVLEVQSRTNFIKRIIILDTVENIHGCESLPNFISRYSDGNIANFKPLHFDPVEQVAAILCSSGTTGLPKGVMQTHQNICVRLIHALDPRVGTQLIPGVTVLVYLPFFHAFGFSITLGYFMVGLRVIMFRRFDQEAFLKAIQDYEVRSVINVPSVILFLSKSPLVDKYDLSSLRELCCGAAPLAKEVAEVAAKRLNLPGIRCGFGLTESTSANIHSLGDEFKSGSLGRVTPLMAAKIADRETGKALGPNQVGELCIKGPMVSKGYVNNVEATKEAIDDDGWLSGGGGSGGGGSGGGGSGGGGSGDKGTRVFKKASPNGKLTVYLGKRDFVDHIDLVDPVDGVVLVDPEYLKERRVYVTLTCAFRYGREDLDVLGLTFRKDLFVANVQSFPPAPEDKKPLTRLQERLIKKLGEHAYPFTFEIPPNLPCSVTLQPGPEDTGKACGVDYEVKAFCAENLEEKIHKRNSVRLVIRKVQYAPERPGPQPTAETTRQFLMSDKPLHLEASLDKEIYYHGEPISVNVHVTNNTNKTVKKIKISVRQYADICLFNTAQYKCPVAMEEADDTVAPSSTFCKVYTLTPFLANNREKRGLALDGKLKHEDTNLASSTLLREGANREILGIIVSYKVKVKLVVSRGGDVAVELPFTLMHPKPKEEPPHREVPENETPVDTNLIELDTNDDDIVFEDFARQRLKGMKDDKEEEEDGTGSPQLNNR

SEQ ID NO: 27 - protein, 832 aa, Artificial Sequence
  Synthetic Barr2 construct
MVKREKNVIYGPEPLHPLEDLTAGEMLFRALRKHSHLPQALVDVVGDESLSYKEFFEATVLLAQSLHNCGYKMNDVVSICAENNTRFFIPVIAAWYIGMIVAPVNESYIPDELCKVMGISKPQIVFTTKNILNKVLEVQSRTNFIKRIIILDTVENIHGCESLPNFISRYSDGNIANFKPLHFDPVEQVAAILCSSGTTGLPKGVMQTHQNICVRLIHALDPRYGTQLIPGVTVLVYLPFFHAFGFHITLGYFMVGLRVIMFRRFDQEAFLKAIQDYEVRSVINVPSVILFLSKSPLVDKYDLSSLRELCCGAAPLAKEVAEVAAKRLNLPGIRCGFGLTESTSAIIQTLGDEFKSGSLGRVTPLMAAKIADRETGKALGPNQVGELCIKGPMVSKGYVNNVEATKEAIDDDGSGGGGSGGGGSGEKPGTRVFKKSSPNCKLTVYLGKRDFVDHLDKVDPVDGVVLVDPDYLKDRKVFVTLTCAFRYGREDLDVLGLSFRKDLFIATYQAFPPVPNPPRPPTRLQDRLLRKLGQHAHPFFFTIPQNLPCSVTLQPGPEDTGKACGVDFEIRAFCAKSLEEKSHKRNSVRLVIRKVQFAPEKPGPQPSAETTRHFLMSDRSLHLEASLDKELYYHGEPLNVNVHVTNNSTKTVKKIKVSVRQYADICLFSTAQYKCPVAQLEQDDQVSPSSTFCKVYTITPLLSDNREKRGLALDGKLKHEDTNLASSTIVKEGANKEVLGILVSYRVKVKLVVSRGGDVSVELPFVLMHPKPHDHIPLPRPQSAAPETDVPVDTNLIEFDTNYATDDDIVFEDFARLRLKGMKDDDYDDQLC

SEQ ID NO: 28 - protein, 574 aa, Artificial Sequence
  Synthetic fusion peptide
MKTIIALSYIFCLVFADYKDDDDAMESPIQIFRGDPGPTCSPSACLLPNSSSWFPNWAESDSNGSVGSEDQQLESAHISPAIPVIITAVYSVVFVVGLVGNSLVMFVIIRYTKMKTATNIYIFNLALADALVTTTMPFQSAVYLMNSWPFGDVLCKIVISIDYYNMFTSIFTLTMMSVDRYIAVCHPVKALDFRTPLKAKIINICIWLLASSVGISAIVLGGTKVREDVDVIECSLQFPDDEYSWWDLFMKICVFVFAFVIPVLIIIVCYTLMILRLKSVRLLSGSREKDRNLRRITKLVLVVVAVFIICWTPIHIFILVEALGSTSHSTAALSSYYFCIALGYTNSSLNPVLYAFLDENFKRCFRDFCFPIKMRMERQSTNRVRNTVQDPASMRDVGGMNKPVGSSGGGGSGGGGSGGGGSGGGGSKGYVNNVEATKEAIDDDGWLHSGDFGYYDEDEHFYVVDRYKELIKYKGSQVAPAELEEILLKNPCIRDVAVVGIPDLEAGELPSAFVVKQPGKEITAKEVYDYLAERVSHTKYLRGGVRFVDSIPRNVTGKITRKELLKQLLEKAGG

SEQ ID NO: 29 - protein, 613 aa, Artificial Sequence
  Synthetic fusion peptide
MDPLNLSWYDDDLERQNWSRPFNGSDGKADRPHYNYYATLLTLLIAVIVFGNVLVCMAVSREKALQTTTNYLIVSLAVADLLVATLVMPWVVYLEVVGEWKFSRIHCDIFVTLDVMMCTASILNLCAISIDRYTAVAMPMLYNTRYSSKRRVTVMISIVWVLSFTISCPLLFGLNNADQNECIIANPAFVVYSSIVSFYVPFIVTLLVYIKIYIVLRRRRKRVNTKRSSRAFRAHLRAPLKGNCTHPEDMKLCTVIMKSNGSFPVNRRRVEAARRAQELEMEMLSSTSPPERTRYSPIPPSHHQLTLPDPSHHGLHSTPDSPAKPEKNGHAKDHPKIAKIFEIQTMPNGKTRTSLKTMSRRKLSQQKEKKATQMLAIVLGVFIICWLPFFITHILNIHCDCNIPPVLYSAFTWLGYVNSAVNPIIYTTFNIEFRKAFLKILHCGSSGGGGSGGGGSGGGGSGGGGSKGYVNNVEATKEAIDDDGWLHSGDFGYYDEDEHFYVVDRYKELIKYKGSQVAPAELEEILLKNPCIRDVAVVGIPDLEAGELPSAFVVKQPGKEITAKEVYDYLAERVSHTKYLRGGVRFVDSIPRNVTGKITRKELLKQLLEKAGG

SEQ ID NO: 30 - protein, 557 aa, Artificial Sequence
  Synthetic fusion peptide
MYPYDVPDYAYPYDVPDYAYPYDVPDYADILNSSTEDGIKRIQDDCPKAGRHNYIFVMIPTLYSIIFVVGIFGNSLVVIVIYFYMKLKTVASVFLLNLALADLCFLLTLPLWAVYTAMEYRWPFGNYLCKIASASVSFNLYASVFLLTCLSIDRYLAIVHPMKSRLRRTMLVAKVTCIIIWLLAGLASLPAIIHRNVFFIENTNITVCAFHYESQNSTLPIGLGLTKNILGFLFPFLIILTSYTLIWKALKKAYEIQKNKPRNDDIFKIIMAIVLFFFFSWIPHQIFTFLDVLIQLGIIRDCRIADIVDTAMPITICIAYFNNCLNPLFYGFLGKKFKRYFLQLLKYIPPKAKSHSNLSTKMSTLSYRPSDNVSSSTKKPAPCFEVEGSSGGGGSGGGGSGGGGSGGGGSKGYVNNVEATKEAIDDDGWLHSGDFGYYDEDEHFYVVDRYKELIKYKGSQVAPAELEEILLKNPCIRDVAVVGIPDLEAGELPSAFVVKQPGKEITAKEVYDYLAERVSHTKYLRGGVRFVDSIPRNVTGKITRKELLKQLLEKAGG